(19) United States
(12) Patent Application Publication
(10) Pub. No.: US 2026/0108604 A1
(43) Pub. Date: Apr. 23, 2026

(54) CAR T CELL COMPOSITIONS FOR TREATMENT OF CANCER

(71) Applicant: Washington University, St. Louis, MO (US)

(72) Inventor: Nathan SINGH, St. Louis, MO (US)

(73) Assignee: Washington University, St. Louis, MO (US)

(21) Appl. No.: 19/111,411

(22) PCT Filed: Sep. 15, 2023
(86) PCT No.: PCT/US23/74347

§ 371 Date: Mar. 13, 2025

Related U.S. Application Data
(60) Provisional application No. 63/407,038, filed on Sep. 15, 2022.
(60) Provisional application No. 63/481,446, filed on Jan. 25, 2023.

(51) Int. Cl. A61K 40/11; A61K 40/31; A61K 40/42; A61P 35/00; C07K 16/28; C12N 9/22; C12N 15/11; C12N 15/90

(52) U.S. Cl. CPC A61K 40/11; A61K 40/31; A61K 40/4212; A61P 35/00; C07K 16/2803; C12N 9/226; C12N 15/11; C12N 15/907; A61K 2239/22; C12N 2310/20

(57) ABSTRACT

Compositions for the treatment of cancer that include CAR T cells in which expression of FOXO3 expression is silenced are disclosed in various aspects. In various other aspects, methods for preventing or delaying a development of dysfunction in a CAR T cell associated with chronic antigen stimulation are described that include silencing the expression of FOXO3 by the CAR T cell.

### CROSS-REFERENCE TO RELATED APPLICATIONS

This application claims priority from U.S. Provisional Application Ser. No. 63/407,038 filed on 15 Sep. 2022 and 63/481,446 filed on 25 Jan. 2023, which are incorporated herein by reference in their entireties.

### STATEMENT REGARDING FEDERALLY SPONSORED RESEARCH OR DEVELOPMENT

This invention was made with government support under CA237740 awarded by the National Institutes of Health. The government has certain rights in the invention.

### MATERIAL INCORPORATED-BY-REFERENCE

Not applicable. The Sequence Listing, which is a part of the present disclosure, includes a computer-readable form comprising nucleotide and/or amino acid sequences of the present invention (file name “020330-WO_Sequence_Listing.xml” created on 8 Sep. 2023; 4,565 bytes) The subject matter of the Sequence Listing is incorporated herein by reference in its entirety.

### FIELD

The present disclosure generally relates to CAR T cell compositions for use in cancer therapy and methods of preventing or delaying the development of dysfunction of CAR T cells associated with chronic antigen stimulation.

### SUMMARY

In various aspects, CAR T cell compositions for use in cancer therapy and the manipulation of FOXO transcription factor and BACH2 expression to prevent or delay the development of dysfunction of CAR T cells associated with chronic antigen stimulation are disclosed herein.

In one aspect of the present disclosure, a composition for the treatment of cancer is provided. The composition comprises a modified chimeric antigen receptor (CAR) T cell, wherein the modified CAR T cell expresses a reduced level of a FOXO transcription factor compared to an unmodified CAR T cell. In some embodiments, the FOXO transcription factor is FOXO1, FOXO3, or FOXO4. In some embodiments, the modified CAR T cell comprises a CD28 signaling domain or a 41BB signaling domain. In some embodiments, the composition further comprises a FOXO inhibitor. In some embodiments, the modified CAR T cell is CRISPR edited to disrupt a FOXO transcription factor gene.

In another aspect of the present disclosure, a method of treating cancer in a subject in need thereof is provided. The method comprises administering to the subject an effective amount of a composition comprising a modified chimeric antigen receptor (CAR) T cell, wherein the modified CAR T cell expresses a reduced level of a FOXO transcription factor compared to an unmodified CAR T cell. In some embodiments, the subject has leukemia or lymphoma.

In yet another aspect of the present disclosure, a method for producing modified CAR T cells expressing a reduced level of a FOXO transcription factor compared to an unmodified CAR T cell is provided. The method comprises: culturing a population of CAR T cells; introducing a composition to the population of CAR T cells that disrupts a FOXO transcription factor gene, yielding a population of modified CAR T cells; and expanding the population of modified CAR T cells. In some embodiments, the composition comprises a Cas protein and a single guide RNA (sgRNA) targeted to FOXO3. In some embodiments, the population of CAR T cells is further modified to express a CD28 or 41BB signaling domain.

In a further aspect of the present disclosure, a composition for the treatment of cancer is provided. The composition comprises a modified chimeric antigen receptor (CAR) T cell, wherein the modified CAR T cell expresses an increased level of BACH2 compared to an unmodified CAR T cell. In some embodiments, the modified CAR T cell comprises a CD28 signaling domain. In some embodiments, the modified CAR T cell is a CD22-targeting CAR T cell.

In a yet a further aspect of the present disclosure, a method of treating cancer in a subject is provided. The method comprises administering to the subject an effective amount of a composition comprising a modified chimeric antigen receptor (CAR) T cell, wherein the modified CAR T cell expresses an increased level of BACH2 compared to an unmodified CAR T cell. In some embodiments, the modified CAR T cell comprises a CD28 signaling domain. In some embodiments, the modified CAR T cell is a CD22-targeting CAR T cell.

In an additional aspect of the present disclosure, a method for producing modified CAR T cells expressing an increased level of BACH2 compared to unmodified CAR T cells is provided. The method comprises: culturing a population of CAR T cells; introducing a first vector comprising a nucleic acid encoding BACH2 to the population of CAR T cells; and expanding the population of T cells. In some embodiments, the method further comprises introducing a second vector comprising a nucleic acid encoding CD28 to the population of CAR T cells. In some embodiments, the first vector and the second vector each comprise a selection marker. In some embodiments, the selection marker is a nucleic acid encoding EGFR or CD34.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a schematic flow of in vitro chronic stimulation assay in accordance with the present disclosure.

FIG. 2 is a graph showing CAR expression in 19/28 and 19/BB after purification before co-culture (day 0) and after 15 days of chronic stimulation (day 15) in accordance with the present disclosure.

FIG. 3A-FIG. 3F is an exemplary embodiment showing chronic CAR stimulation results in T-cell dysfunction in accordance with the present disclosure. FIG. 3A is a line graph showing expansion of 19/28 and 19/BB CAR T cells over the course of chronic stimulation. FIG. 3B is a bar graph showing target Nalm6 cells per CAR T cells over the course of chronic stimulation. FIG. 3C and FIG. 3D include bar graphs showing production of (FIG. 3C) interferon γ and (FIG. 3D) tumor necrosis factor α by 19/28 and 19/BB cells isolated at days 7 and 15 of chronic stimulation cultures upon restimulation. FIG. 3E is a dot plot showing kinetics of dysfunction onset as reflected by first day of failure as measured by T-cell contraction or loss of tumor control. Data from n=4 independent donors. FIG. 3F includes bar graphs showing change in memory phenotype of CD8+ CAR T-cell products after either acute (single combination with Nalm6 cells) or chronic stimulation. Representative data from n=4 independent donors for FIG. 3A-FIG. 3D and FIG. 3F. *P<0.05; **P<0.01; ***P<0.001; ****P<0.0001 using two-tailed analysis of variance (ANOVA) with Bonferroni correction for multiple comparisons.

FIG. 4A-FIG. 4B include bar graphs showing change in memory phenotype of CD4+ CAR T cell products after either acute (single combination with Nalm6 cells) or chronic stimulation (FIG. 4A) and activation of central T cell transcription factors in CAR Jurkat cells engineered to express a triple fluorescent reporter system (FIG. 4B) in accordance with the present disclosure.

FIG. 5 is a line graph showing surface expression of PD1 during chronic stimulation in accordance with the present disclosure. Representative data from n=4 independent donors. *P<0.05; **P<0.01; ***P<0.001; ****P<0.0001 using two-tailed analysis of variance (ANOVA) with Bonferroni correction for multiple comparisons.

FIG. 6A-FIG. 6B include tSNE projections of 19/28 (FIG. 6A) and 19/BB cells (FIG. 6B) evaluated by CyTOF in accordance with the present disclosure.

FIG. 7 is a heat map of median signal intensities from all cytometry by time of flight markers evaluated in accordance with the present disclosure.

FIG. 8A-FIG. 8C include tsNE projections of expression of PD1 (FIG. 8A) TIGIT (FIG. 8B) and CD62L (FIG. 8C) in 19/28 and 19/BB cells in accordance with the present disclosure.

FIG. 9 includes violin plots of normalized signal intensity for T-cell markers on day-15 cells in accordance with the present disclosure. Data representative of n=2 donors.

FIG. 10 is a PCA plot of RNAseq by donor (n=1 donor for day 0, n=4 donors for days 6 and 15) in accordance with the present disclosure.

FIG. 11 shows principal component analysis of bulk RNAseq of 19/28 and 19/BB cells on days 0, 6, and 15 in accordance with the present disclosure.

FIG. 12A-FIG. 12B include volcano plots of DEGs at day 0 (FIG. 12A) and day 6 (FIG. 12B) in accordance with the present disclosure.

FIG. 13A-FIG. 13C is an exemplary embodiment showing chronic activation of costimulatory domains directs distinct genomic activity in accordance with the present disclosure. FIG. 13A is a heatmap of DEGs between day-15 19/28 cells and day-15 19/BB cells. FIG. 13B is a volcano plot of day-15 DEGs. n=1 donor for day-0 samples and n=4 donors for days 6 and 15, all performed in technical triplicates. FIG. 13C includes line graphs showing normalized transcript counts of key exhaustion markers over time. Significance determined using two-way ANOVA. ****P<0.0001.

FIG. 14A-FIG. 14E is an exemplary embodiment in accordance with the present disclosure. FIG. 14A is a plot showing KEGG pathways enriched in dysfunctional 19/28 and 19/BB cells FIG. 14B, FIG. 14C, FIG. 14D, and FIG. 14E include Venn diagrams showing overlap of genes with higher expression in 19/28 and 19/BB with previously published gene sets defining exhausted tumor-infiltrating lymphocytes. Significance determined using Fisher's Exact Test.

FIG. 15A-FIG. 15C is an exemplary embodiment showing chronic activation of costimulatory domains directs distinct genomic activity in accordance with the present disclosure. FIG. 15A-FIG. 15B include gene set enrichment analysis of genes present in >2 of 4 gene sets (FIG. 15A) and all 4 human TIL exhaustion gene sets (FIG. 15B). FIG. 15C is a heatmap of day-15 sample expression of genes present in all 4 human TIL exhaustion gene sets.

FIG. 16 is a PCA plot of ATACseq by donor (n=2 donors) in accordance with the present disclosure.

FIG. 17A-FIG. 17D is an exemplary embodiment showing chronic activation of costimulatory domains directs distinct genomic activity in accordance with the present disclosure. FIG. 17A shows principal component analysis of ATAC sequencing of 19/28 and 19/BB cells at days 0, 6, and 15. n=2 donors for all time points performed in technical triplicates. FIG. 17B is a graph showing gene tracks at transcriptional start sites of PDCD1 and TOX2 reflecting chromatin accessibility.

FIG. 17C is a graph showing correlated transcript count and transcriptional start site accessibility for PDCD1 over time. FIG. 17D is a Venn diagram showing overlap of genes with increased accessibility in exhausted T cells (as defined in Brinkman et al. (2014) Nucleic Acids Res. 42(22):3168) and genes with increased accessibility in dysfunctional 19/28 or 19/BB cells.

FIG. 18 is a plot showing uniform manifold approximation and projection (UMAP) of 19/28 and 19/BB cells over time in accordance with the present disclosure.

FIG. 19A-FIG. 19B is an exemplary embodiment in accordance with the present disclosure. FIG. 19A includes line graphs showing proportion of each sample (19/28 or 19/BB at day 0, 6 or 15) contained within each cluster. FIG. 19B includes bar graphs showing KEGG pathways enriched in each cluster.

FIG. 20A-FIG. 20C is an exemplary embodiment showing single-cell analysis reveals that dysfunctional 41BB CAR T cells are a unique terminal state in accordance with the present disclosure. FIG. 20A includes pie charts showing proportion of each cluster present in each sample. FIG. 20B is a plot showing CD8A expression in each cluster. FIG. 20C is a UMAP plot showing reclustering of CD8A-expressing 19/28 and 19/BB cells from days 0, 6, and 15.

FIG. 21 includes pie charts showing the proportion of each CD8 cluster in day 15 samples in accordance with the present disclosure.

FIG. 22A-FIG. 22C is an exemplary embodiment showing single-cell analysis reveals that dysfunctional 41BB CAR T cells are a unique terminal state in accordance with the present disclosure. FIG. 22A is a plot showing pseudotime analysis of CD8 cells. FIG. 22B is a plot showing mapping of CD8 clusters onto pseudotime. FIG. 22C is a bar graph showing proportion of each sample contained in terminal CD8 clusters 0 and 1.

FIG. 23 includes Venn diagrams showing approach to generate a signature of 41BB-driven CAR T cell dysfunction in accordance with the present disclosure. Genes that were uniquely upregulated in day 15 (dysfunctional) 19/BB cells by bulk RNAseq were compared to genes that were uniquely upregulated in cluster 8 from the scRNAseq dataset. Genes that were shared from these two lists were used to generate the 41BB dysfunction signature of 145 genes. Filtering to identify genes in both datasets was performed with FDR<0.05 and log₂-fold change>1.5.

FIG. 24A-FIG. 24G is an exemplary embodiment showing failing CAR T cells express a dysfunctional 41BB CAR T-cell signature in accordance with the present disclosure. FIG. 24A is a schematic showing peripheral blood cells from a patient who received tisagenlecleucel for diffuse large B-cell lymphoma were collected 14 and 100 days after infusion and purified for CAR-expressing cells. FIG. 24B is a UMAP plot of day-14 and day-100 cells. FIG. 24C includes UMAP plots of expression of CD4 and CD8A in peripheral blood CAR T cells. FIG. 24D is a Venn diagram showing overlap between in vitro-defined signature of 41BB-driven dysfunction and genes defining day-100 cell identity. Significance of overlap determined using Fisher exact test. FIG. 24E includes violin plots showing expression of top 10 driver genes in day-14 and day-100 cells. FIG. 24F is a heat map showing log₂ fold change in expression of master exhaustion genes in day-100 cells compared with that in day-14 cells. FIG. 24G includes Venn diagrams showing overlap between genes defining day-100 cell identity and TIL exhaustion signatures. Significance of overlap determined using Fisher exact test.

FIG. 25A-FIG. 25E is an exemplary embodiment showing TBBD cells demonstrate reactivation of FOXO3 in accordance with the present disclosure. FIG. 25A and FIG. 25B include transcription factor motif analysis demonstrating increased accessibility of (FIG. 25A) AP1 sites in 19/28 cells and (FIG. 25B) HOX and FOX sites in 19/BB cells as cells progress from resting to dysfunctional. FIG. 25C and FIG. 25D show pathway enrichment analysis of unique sites with increased accessibility in (FIG. 25C) 19/28 cells and (FIG. 25D) 19/BB cells. FIG. 25E includes bar graphs showing expression of FOXO1, FOXO3, and FOXO4 transcripts over time. Significance determined using two-way ANOVA.

FIG. 26A-FIG. 26B include bar graphs showing expression of FOXP transcripts over time (FIG. 26A) and expression of bZIP/AP1 factors over time (FIG. 26B) in accordance with the present disclosure.

FIG. 27A-FIG. 27B is an exemplary embodiment showing T_{BBD} cells demonstrate reactivation of FOXO3 in accordance with the present disclosure. FIG. 27A is a UMAP plot showing expression of the FOXO3 regulon in 19/28 and 19/BB cells collected on days 0, 6, and 15. FIG. 27B includes graphs showing FOXO3 regulon score for each scRNAseq cluster (top) and normalized FOXO3 transcript count for each cluster (bottom).

FIG. 28A-FIG. 28C is an exemplary embodiment in accordance with the present disclosure. FIG. 28A is a graph showing enrichment of FOXO3 target genes in genes that marked identity of cluster 8. FIG. 28B is a line graph showing expression of FOXP3 transcripts over time in chronically stimulated 19/28 and 19/BB cells. FIG. 28C is includes a UMAP plot showing expression of FOXP3 in scRNAseq of 19/28 and 19/BB cells collected at day 0, 6 and 15 and a table representing log 2-fold change (LFC) in expression of FOXP3 in each cluster that it is found to be enriched with associated false discovery rate (FDR).

FIG. 29 is a dot plot showing expression of FOXO3 regulon in day-14 and day-100 cells collected from patient peripheral blood in accordance with the present disclosure. Significance determined using Mann-Whitney test.

FIG. 30 is a schematic representation of FOXO3^{KO} cells being evaluated in accordance with the present disclosure.

FIG. 31A-FIG. 31E in an exemplary embodiment in accordance with the present disclosure. FIG. 31A shows sequencing analysis of genomic FOXO3 in CAR T cell manufacturing products demonstrating high-efficiency knockout performed using Synthego ICE (including SEQ ID NO: 1, SEQ ID NO: 2, SEQ ID NO: 3, and SEQ ID NO: 4). FIG. 311B-FIG. 31C include bar graphs showing T cell expansion during manufacturing of 19/28 (FIG. 31B) or 19/BB cells (FIG. 31C) with genomic disruption of FOXO3 (N=3 independent donors). FIG. 31D-FIG. 31E include line graphs showing expansion of FOXO3^{KO} or WT 19/28 (FIG. 31D) or 19/BB CAR T cells (FIG. 31E) during chronic stimulation. Representative data from n=3 independent donors. Statistical significance determined by two-tailed ANOVA with Bonferroni correction for multiple comparisons.

FIG. 32A-FIG. 32C is an exemplary embodiment showing manipulation of FOXO3 affects 41BB-based CAR T-cell function in accordance with the present disclosure. FIG. 32A includes bar graphs showing target Nalm6 cells per CAR T cells over the course of chronic stimulation. Representative data from n=3 donors. FIG. 32B includes dot plots showing day of dysfunction onset as measured based on the first day of T-cell contraction or loss of tumor control. Data from n=3 independent donors. FIG. 32C is a schematic representation of FOXO3^{OE} cells being evaluated.

FIG. 33A-FIG. 33E is an exemplary embodiment in accordance with the present disclosure. FIG. 33A is a Western blot of lysates from CAR T cells engineered to overexpress FOXO3. FIG. 33B is a bar graph showing T cell expansion during manufacturing of 19/28 and 19/BB with overexpression of FOXO3. N=2 independent donors. FIG. 33C and FIG. 33D include line graphs showing expansion of FOXO3^{0E} 19/28 (FIG. 33C) or 19/BB (FIG. 33D) CART cells during chronic stimulation. Representative data from n=2 independent donors. FIG. 33E is a line graph showing expression of 7AAD by CAR+ T cells.

FIG. 34A-FIG. 34B is an exemplary embodiment showing manipulation of FOXO3 affects 41BB-based CAR T-cell function in accordance with the present disclosure. FIG. 34A includes bar graphs showing target Nalm6 cells per CAR T cells over the course of chronic stimulation. Representative data from n=3 donors. FIG. 34B is a dot plot showing day of dysfunction onset as measured by first day of T-cell contraction or loss of tumor control. Data from n=3 independent donors.

FIG. 35 is a line graph showing CAR T cell size as measured by forward scatter area over the course of chronic stimulation in accordance with the present disclosure. Representative data from n=3 independent donors. Statistical significance determined by two-tailed ANOVA with Bonferroni correction for multiple comparisons.

FIG. 36A-FIG. 36B is an exemplary embodiment in accordance with the present disclosure. FIG. 36A is a line graph showing Nalm6 progression over time after treatment with 0.125×10⁶ CAR T cells. Radiance curves were stopped at time of first animal death. Significance determined using two-way ANOVA. FIG. 36B includes line graphs showing individual animal radiance over time. For all studies n=5 animals per group.

FIG. 37 is a survival curve showing survival of mice after treatment with 0.125×10⁶ CAR T cells in accordance with the present disclosure. Significance determined using log-rank test, n=5 mice per group.

FIG. 38 is a line graph showing Nalm6 progression overtime aftertreatment with 0.5×10⁶ CAR T cells in accordance with the present disclosure. Radiance curves were stopped at time of first animal death. Significance determined using two-way ANOVA, n=5 animals per group.

FIG. 39 is a survival curve showing survival of mice after treatment with 0.5×10⁶ CAR T cells in accordance with the present disclosure. Significance determined using log-rank test, n=5 mice per group.

FIG. 40A-FIG. 40E is an exemplary embodiment showing antigen-independent signaling of CARs has a different impact on T cell function that is dependent on costimulatory domain in accordance with the present disclosure. FIG. 40A is a schematic showing design of CARs that do or do not signal in the absence of antigen (tonic signaling). FIG. 40B is a line graph showing expansion of CAR T cells engineered to express these constructs demonstrates improved expansion of tonic signaling 41BB-based cells (41BB^{hi}) and suppressed expansion of tonic signaling CD28-based cells (CD28^{hi}). FIG. 40C is a line graph showing co-culture with CD22+ ALL cells demonstrates superior tumor killing by 41BB^{hi} and inferior killing by CD28^{hi} cells. FIG. 40D includes bar graphs showing a similar trend is observed for secretion of effector cytokines IFNg and IL2. FIG. 40E is a line graph showing CD28^{hi} cells maintain high PD1 expression, reflecting an exhausted state.

FIG. 41A-FIG. 41B is an exemplary embodiment showing tonic signaling results in different transcriptional states in T cells bearing either 41BB^{hi} or CD28^{hi} in accordance with the present disclosure. FIG. 41A is a heatmap of differentially expressed genes in CAR T cells at the end of T cell manufacturing reflects that both 41BB^{hi} and CD28^{hi} are more transcriptionally active than their non-tonic signaling counterparts. FIG. 41B includes graphs showing gene set enrichment analysis revealing that genes that are upregulated in 41BB^{hi} cells are targets of BACH2 while genes that are upregulated in CD28^{hi} are targets of NFAT.

FIG. 42A-FIG. 42C is an exemplary embodiment showing engineering human T cells to express independent vectors encoding CAR and BACH2 yields high purity double-positive cells in accordance with the present disclosure. FIG. 42A is a schematic showing engineering approach to enable expression of CAR and BACH2. FIG. 42B-FIG. 42C include graphs showing expression (FIG. 42B) prior to selection and (FIG. 42C) expression following selection using magnetic beads.

FIG. 43A-FIG. 43B is an exemplary embodiment showing introduction of BACH2 rescues dysfunction of tonically signaling CD28^{hi} CAR T cells in accordance with the present disclosure. FIG. 43A is a line graph showing expansion of CAR T cells during manufacturing demonstrates significant improvement in growth of CD28^{hi} cells with inclusion of BACH2. FIG. 43B is a line graph showing BACH2 significantly improves target ALL killing by CD28^{hi} CAR T cells.

### DETAILED DESCRIPTION OF THE INVENTION

The present disclosure is based, at least in part, on the discovery that genetic modifications to CAR T cells can improve their function and mitigate failure of CAR T cells in the treatment of cancer.

Two of the fundamental barriers to the success of chimeric antigen receptor (CAR)-engineered T cells in the treatment of cancer are (1) toxicity in the form of cytokine release syndrome (CRS), thought to result from overly-exuberant T cell activation, and (2) impaired CAR T cell persistence, thought to result from the rapid and aberrant development of T cell dysfunction. Both of these processes are dependent on antigen-dependent CAR-driven intracellular signaling, a process that, by virtue of the simplistic CAR design, is much stronger as well as less organized than that of the native T cell antigen receptor (TCR).

Without being limited to any particular theory, the development of dysfunction in CAR T cells is thought to be driven by chronic antigen stimulation. Dysfunction in CAR T cells may be characterized by a variety of phenomena including, but not limited to, impaired expansion, loss of anti-tumor efficacy, and enhanced cytokine secretion such as IFN-gamma and TNF-alpha secretions.

As described herein, through high-resolution sequencing and in vitro functional assays, it was identified that the transcription factor forkhead family 03 (FOXO3) plays a central role in promoting the dysfunction of 41BB costimulated, but not CD28 costimulated, CAR T cells (see e.g., Example 1). Disruption of the gene encoding this protein significantly improves anti-tumor CAR T cell function and delays the onset of dysfunction that results from chronic antigen exposure.

Further, it was also found that BACH2 overexpression can overcome tonic CAR signaling-induced dysfunction by antagonizing exhaustion programs (see e.g., Example 2).

### Chimeric Antigen Receptor (CAR) Constructs/Therapy

Chimeric Antigen Receptor T-cell therapy is an exciting new mode of cancer therapy in which T cells are engineered to attack tumor cells. Cells are engineered to express chimeric T-cell receptors, which fuse a single chain antibody (scFv) with specificity against a tumor antigen to intracellular signaling modules derived from T-cell signaling proteins.

In various aspects, a composition for the treatment of cancer is described that includes a modified CAR T cell targeted to one or more antigens of tumor cells associated with cancer. A “modified” CAR T cell as described herein refers to a CAR T cell that has been modified to alter gene expression compared to an unmodified CAR T cell. In some embodiments, CAR T cells may be modified to express a reduced level of a FOXO transcription factor, such as FOXO1, FOXO3, or FOXO4, compared to an unmodified CAR T cell. In some embodiments, CAR T cells may be modified to express an increased level of BACH2, compared to an unmodified CAR T cell.

In various aspects, a composition for the treatment of cancer is described that includes a CAR T cell targeted to one or more antigens of tumor cells associated with cancer, in which a sequence encoding a FOXO transcription factor is inactivated. The sequence can be inactivated by any method known in the art, such as by CRISPR. In some embodiments, the sequence is modified so that its expression is reduced by at least about 50, 60, 70, 80, 90, or 95% as compared to the expression in the absence of the gene modification or in the absence of an inhibitor introduced to effect the modification. In some embodiments, expression is reduced by at least about 90%.

In some aspects, the FOXO transcription factor sequence of the CAR T cell is inactivated prior to administering the composition to a patient in need. In some aspects, the CAR T cells are FOXO transcription factor knockout cells, in which the sequence encoding the FOXO transcription factor is deleted from the genome of the CAR T cell. In other aspects, the CAR T cells are FOXO transcription factor knockdown cells, in which a nucleotide-based inhibitor of the FOXO transcription factor is inserted into the genome of the CAR T cell, resulting in inhibition of FOXO transcription factor expression in the CAR T cell without deleting it from the genome. In some aspects, a FOXO transcription factor inhibitor is administered in combination with the FOXO transcription factor knockout cells or FOXO transcription factor knockdown cells.

In other aspects, a CAR T cell that includes an intact FOXO transcription factor sequence may be administered to a patient in need along with a FOXO transcription factor inhibitor to inactivate the FOXO transcription factor produced by the CAR T cells after administration. Any suitable FOXO transcription factor inhibitor may be coadministered without limitation. Non-limiting examples of suitable FOXO transcription factor inhibitors include RNAi compositions, dominant negative FOXO transcription factor, or any other suitable forms of post-transcriptional/post-translational silencing.

In various aspects, a composition for the treatment of cancer is described that includes a CAR T cell targeted to one or more antigens of tumor cells associated with cancer, in which a sequence encoding BACH2 is overexpressed in the CAR T cell. The sequence can be overexpressed by any method known in the art, such as by introducing a genetic vector comprising the sequence to the CAR T cell.

CAR designs are generally tailored to each cell type. The present disclosure is drawn to T cells and can be useful in other immune cell type embodiments.

### FOXO Inhibitor

One aspect of the present disclosure provides for a FOXO inhibitor. FOXO transcription factors are a class of evolutionarily conserved molecules important to a number of biological processes (see e.g., Link (2019) Methods Mol Biol. 1890:1-9). The mammalian class includes four members, FOXO1, FOXO3, FOXO4 and FOXO6. The present disclosure provides compositions or methods for treating cancer based on the discovery that disrupting the FOXO3 gene significantly improves anti-tumor CAR T cell function and delays the onset of dysfunction that results from chronic antigen exposure.

As described herein, inhibitors or antagonists of FOXO (e.g., antibodies, fusion proteins, small molecules) can improve CAR T cell function. A FOXO inhibitor can be any agent that can inhibit FOXO activity or signaling, downregulate FOXO protein level or expression, or knockdown FOXO gene expression.

For example, the FOXO inhibitor can be an anti-FOXO antibody. As an example, the anti-FOXO antibody can be anti-FOXO1 antibody, an anti-FOXO3 antibody, or an anti-FOXO4 antibody. Furthermore, the anti-FOXO antibody can be a murine antibody, a humanized murine antibody, or a human antibody.

As another example, a FOXO inhibitor can be carbenoxolone (CBX), which has been shown to be a potent and specific inhibitor of FOXO3 (see e.g., Salcher et al. (2019) Oncogene. 39(5):1080-1097).

As another example, a FOXO inhibitor can be 1-(4,6-dimethylpyrimidin-2-yl)-3-(4-propoxyphenyl)guanidine or its oxalate salt, which have been shown to be potent and specific inhibitors of FOXO3 (see e.g., Hagenbuchner et al. (2019) eLife. 8: e48876).

As another example, a FOXO inhibitor can be 5-amino-7-(cyclohexylamino)-1-ethyl-6-fluoro-4-oxo-1,4-dihydroquinoline-3-carboxylic acid (AS1842856), which has been shown to be potent and specific inhibitor of FOXO1 (see e.g., Nagashima et al. (2010) Mol Pharmacol. 78(5):961-70).

As another example, a FOXO inhibitor can be JY-2 (5-(2,4-dichlorophenyl)-3-(pyridin-2-yl)-1,2,4-oxadiazole), which has been shown to be a potent and specific inhibitor of FOXO1 (see e.g., Choi et al. (2021) Eur J Pharmacol. 899:174011).

As another example, a FOXO inhibitor can be an inhibitory protein that antagonizes a FOXO transcription factor.

As another example, a FOXO inhibitor can be a short hairpin RNA (shRNA) or a short interfering RNA (siRNA) targeting a FOXO gene.

As another example, a FOXO inhibitor can be a single guide RNA (sgRNA) targeting a FOXO gene.

Inhibiting FOXO can be performed by genetically modifying a FOXO gene in a subject or genetically modifying a subject to reduce or prevent expression of a FOXO gene, such as through the use of CRISPR-Cas9 or analogous technologies, wherein, such modification reduces or prevents expression or activity of a FOXO transcription factor.

Inhibition of agents as described herein can be determined by standard pharmaceutical procedures in assays or cell cultures for determining the IC₅₀. The half maximal inhibitory concentration (IC₅₀) is a measure of the potency of a substance in inhibiting a specific biological or biochemical function. The IC₅₀ is a quantitative measure that indicates how much of a particular inhibitory substance (e.g., pharmaceutical agent or drug) is needed to inhibit, in vitro, a given biological process or biological component by 50%. Depending on the embodiment, the biological component is an enzyme, cell, cell receptor, or microorganism, for example. IC₅₀ values are typically expressed as molar concentration. IC₅₀ is generally used as a measure of antagonist drug potency in pharmacological research. IC₅₀ is comparable to other measures of potency, such as EC₅₀ for excitatory drugs. EC₅₀ represents the dose or plasma concentration required for obtaining 50% of a maximum effect in vivo. IC₅₀ can be determined with functional assays or with competition binding assays.

### Cancer

Methods and compositions as described herein can be used for the prevention, treatment, or slowing of the progression of cancer or tumor growth. For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sézary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor. Brain or spinal cord tumors can be acoustic neuroma; astrocytoma, atypical teratoid rhabdoid tumor (ATRT); brain stem glioma; chordoma; chondrosarcoma; choroid plexus; CNS lymphoma; craniopharyngioma; cysts; ependymoma; ganglioglioma; germ cell tumor; glioblastoma (GBM); glioma; hemangioma; juvenile pilocytic astrocytoma (JPA); lipoma; lymphoma; medulloblastoma; meningioma; metastatic brain tumor; neurilemmomas; neurofibroma; neuronal & mixed neuronal-glial tumors; non-Hodgkin lymphoma; oligoastrocytoma; oligodendroglioma; optic nerve glioma; pineal tumor; pituitary tumor; primitive neuroectodermal (PNET); rhabdoid tumor; or schwannoma. An astrocytoma can be grade I pilocytic astrocytoma, grade II—low-grade astrocytoma, grade III anaplastic astrocytoma, grade IV glioblastoma (GBM), or a juvenile pilocytic astrocytoma. A glioma can be a brain stem glioma, ependymoma, mixed glioma, optic nerve glioma, or subependymoma.

### Molecular Engineering

In some aspects, the manipulation of FOXO transcription factor or BACH2 expression in the CAR T cells may be implemented using molecular engineering methods.

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as an enhancer and/or promoter regions and lead to efficient transcription of the gene carried on the expression vector. The goal of a well-designed expression vector is the efficient production of protein, and this may be achieved by the production of a significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as: (i) by disabling repressors. The gene is expressed because an inducer binds to the repressor. The binding of the inducer to the repressor prevents the repressor from binding to the operator. RNA polymerase can then begin to transcribe operon genes; or (ii) by binding to activators. Activators generally bind poorly to activator DNA sequences unless an inducer is present. An activator binds to an inducer and the complex binds to the activation sequence and activates a target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as many as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences. In addition to T2A, F2A, P2A, or E2A can be used.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed”, “transgenic”, and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein are within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted, or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes fora different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6 (log₁₀[Na⁺])+0.41 (fraction G/C content)−0.63 (% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged | K R H |
| (Basic): | |
| Negatively Charged | D E |
| (Acidic): | |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tₘ of specific internal domains of the siRNA, siRNA length, position of the target sequence within the CDS (coding region), and nucleotide content of the 3′ overhangs.

### Genome Editing

In some aspects, the manipulation of FOXO transcription factor or BACH2 expression in the CAR T cells may be implemented using genome editing methods.

As described herein, gene editing was used to disrupt the expression of the cell's endogenous FOXO3. Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of endogenous FOXO transcription factor expression or enhancement of BACH2 expression by genome editing can result in enhanced T cell function. Beyond enhancing the efficiency of receptor pairing, this also enables an allogeneic cell therapy product, a highly-sought goal in the development of cell-based immunotherapies.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for CART cell engineering by the removal of endogenous FOXO transcription factor expression or the enhancement of BACH2 expression.

### T Cell Engineering

In some aspects, lentiviral-based or CRISPR-based gene editing may be used to disrupt the expression of the cell's endogenous FOXO transcription factor or factors or increase the cell's expression of BACH2.

Any vector known in the art can be used. For example, the vector can be a viral vector selected from retrovirus, lentivirus, herpes, adenovirus, adeno-associated virus (AAV), rabies, Ebola, lentivirus, or hybrids thereof. As another example, non-viral vectors can be used including plasmid DNA (pDNA) or RNAi.

Genetic modification can be performed either ex vivo or in vivo. The ex vivo strategy is based on the modification of cells in culture and transplantation of the modified cell into a patient.

The use of endonucleases for targeted genome editing can utilize these enzymes as custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents ortogether with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing cancer or tumor progression in a subject in need of administration of a therapeutically effective amount of a composition comprising a modified CAR T cell, so as to substantially inhibit cancer growth or proliferation, slow the progress of cancer growth or proliferation, or limit the development of cancer, and further preventing or delaying the development of dysfunction of the CAR T cells associated with chronic antigen stimulation.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a composition comprising a modified CAR T is, for example, an amount that would cause the desired therapeutic effect in a subject while minimizing undesired side effects. In various embodiments, an effective amount of a composition comprising a modified CAR T cell described herein can substantially inhibit cancer growth or proliferation, slow the progress of cancer growth or proliferation, or limit the development of cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a composition comprising a modified CAR T cell can be employed in pure form or, where such forms exist, in pharmaceutically acceptable salt form and with or without a pharmaceutically acceptable excipient. For example, the compositions of the present disclosure can be administered, at a reasonable benefit/risk ratio applicable to any medical treatment, in a sufficient amount to substantially inhibit cancer growth or proliferation, slow the progress of cancer growth or proliferation, or limit the development of cancer.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from the compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a composition comprising a modified CAR T cell can occur as a single event or over a time course of treatment. For example, a composition comprising a modified CAR T cell can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer treatment.

### Immunotherapy

As described herein, the provided compositions and methods allow for the treatment of cancer with a CAR T cell with a modified CAR T cell. Immunotherapies are a new generation of cancer therapy that has revolutionized the treatment of otherwise terminal cancers, often achieving durable, sustained remission in cancers that were otherwise thought to be refractory to standard first- and second-line therapies. Thousands of patients annually are now treated with these life-saving therapies.

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to affect cell killing. The antibody also may be conjugated to a drug or toxin (chemotherapeutic, radionuclide, ricin A chain, cholera toxin, pertussis toxin, etc.) or serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Examples of immunotherapy can be immune effector cell (IEC) therapy or T cell engaging therapy (e.g., CD19-specific T cell engager, such as blinatumomab, T cell engaging monoclonal antibody, bispecific T cell engager (BiTE) therapy).

In some embodiments, the provided methods are used before, after, or in concurrence with any form of BsMAb therapy. For example, the BsMAb therapy can be any one or more of the currently FDA-approved BsMAb therapies, such as blinatumomab, emicizumab, or amivantamab.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate-specific antigen, urinary tumor-associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including cytokines such as IL-2, IL-4, IL-12, GM-CSF, γ-IFN, chemokines such as MIP-1, MCP-1, IL-8, and growth factors such as FLT3 ligand. Combining immune-stimulating molecules, either as proteins or using gene delivery in combination with a tumor suppressor has been shown to enhance anti-tumor effects (Ju et al., 2000). Moreover, antibodies against any of these compounds may be used to target the T cells to the cancer target.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF, TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945), and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

In some embodiments, the immunotherapy in accordance with the present disclosure is CAR T cell therapy in which the CAR T cells are modified to silence FOXO transcription factor expression or increase BACH2 expression. Generally, CAR T cell therapy refers to any type of immunotherapy in which a subject's T cells are genetically modified to express chimeric antigen receptors. These chimeric antigen receptors allow the T cells to more effectively recognize and subsequently destroy cancer cells. Typically, T cells are first harvested from a subject, genetically altered to express a CAR targeting an antigen of interest (e.g., an antigen expressed on the surface of a tumor or cancer cell), and then infused back into the subject. Once infused into the subject, CAR T cells bind to the target antigen and are activated, allowing them to proliferate and become cytotoxic. As described herein, the CAR T cells may be further genetically altered to silence FOXO transcription factor expression, or alternatively, the CAR T cells may be coadministered with a FOXO transcription factor inhibitor configured to silence expression of a FOXO transcription factor post-transcriptionally or post-translationally. CAR T cells may also be genetically altered to increase BACH2 expression.

### Checkpoint Immunotherapy

The constructs and methods described herein can be used in combination with checkpoint immunotherapy. An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response.

Cancer cells can find ways to use these checkpoints to avoid being attacked by the immune system. But drugs that target these checkpoints hold a lot of promise as a cancer treatment. These drugs are called checkpoint inhibitors. Checkpoint inhibitors used to treat cancer don't work directly on the tumor at all. They only take the brakes off an immune response that has begun but has not yet been working at its full force.

Checkpoint immunotherapy has been extensively shown to unleash T cell effector functions to control tumors in many cancer patients. However, tumor cells can evade immunological elimination by recruiting myeloid cells that induce an immunosuppressive state. Recent high-dimensional profiling studies have shown that tumor-infiltrating myeloid cells are considerably heterogeneous, and may include both immunostimulatory and immunosuppressive subsets, although they do not fit the M1/M2 paradigm. Thus, depletion of suppressive myeloid cells from tumors, blockade of their functions, or induction of myeloid cells with immunostimulatory properties may provide important approaches for improving immunotherapy strategies, perhaps in synergy with checkpoint blockade.

Any immune checkpoint inhibitor known in the art can be used. For example, a PD-1 inhibitor can be used. These drugs are typically administered IV (intravenously). PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it tells the T cell to leave the other cell alone. Some cancer cells have large amounts of PD-L1, which helps them hide from an immune attack.

Monoclonal antibodies that target either PD-1 or PD-L1 can block this binding and boost the immune response against cancer cells. These drugs have shown a great deal of promise in treating certain cancers.

Examples of drugs that target PD-1 can include Pembrolizumab (Keytruda), Nivolumab (Opdivo), or Cemiplimab (Libtayo). These drugs have been shown to be helpful in treating several types of cancer, and new cancer types are being added as more studies show these drugs to be effective.

As another example, a PD-L1 inhibitor can be used. Examples of drugs that target PD-L1 can include Atezolizumab (Tecentriq), Avelumab (Bavencio), or Durvalumab (Imfinzi). These drugs have also been shown to be helpful in treating different types of cancer, and are being studied for use against others.

CTLA-4 is another protein on some T cells that acts as a type of “off switch” to keep the immune system in check. For example, Ipilimumab (Yervoy) is a monoclonal antibody that attaches to CTLA-4 and reduces or blocks its function. This can boost the body's immune response against cancer cells. This drug can be used to treat melanoma of the skin and other cancers.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation have gained significant interest by researchers as a potential new therapeutic strategy for a wide range of diseases, in particular for degenerative and immunogenic pathologies.

Allogeneic cell therapy or allogeneic transplantation uses donor cells from a different subject than the recipient of the cells. A benefit of an allogeneic strategy is that unmatched allogeneic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation use cells from another species. For example, pig-derived cells can be transplanted into humans. Xenogeneic cell therapies can involve human cell transplantation into experimental animal models for assessment of efficacy and safety or enable xenogeneic strategies for humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to the CAR T cells, modified CAR T cells, FOXO inhibitors, Cas protein, sgRNAs, components, constructs, pairs, and plasmids. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification is to be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it is to be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Costimulatory Domains Direct Distinct Fates of Car-Driven T-Cell Dysfunction

This Example describes the costimulatory domains that are critical regulators of CAR-driven T-cell failure and targeted interventions to overcome costimulation-dependent dysfunctional programs.

T cells engineered to express chimeric antigen receptors (CARs) targeting CD19 have demonstrated impressive activity against relapsed or refractory B-cell cancers yet fail to induce durable remissions for nearly half of all patients treated. Enhancing the efficacy of this therapy requires detailed understanding of the molecular circuitry that restrains CAR-driven antitumor T-cell function. Described herein is an in vitro model developed and validated that drives T-cell dysfunction through chronic CAR activation and how CAR costimulatory domains, central components of CAR structure and function, contribute to T-cell failure was investigated. Chronic activation of CD28-based CARs was found to result in activation of classical T-cell exhaustion programs and development of dysfunctional cells that bear the hallmarks of exhaustion. In contrast, 41BB-based CARs activate a divergent molecular program and direct differentiation of T cells into a novel cell state. Interrogation using CAR T cells from a patient with progressive lymphoma confirmed the activation of this novel program in a failing clinical product. Furthermore, 41BB-dependent activation of the transcription factor FOXO3 was demonstrated to be directly responsible for impairing CAR T-cell function. These findings identify that costimulatory domains are critical regulators of CAR-driven T-cell failure and that targeted interventions can overcome costimulation-dependent dysfunctional programs.

### Introduction

Failure of chimeric antigen receptor (CAR)-engineered T cells against both hematologic and solid cancers most often results from defects in T-cell function. This manifests as poor CAR T-cell expansion, limited persistence, and ineffective antitumor cytotoxicity. Correlative studies have identified an association between high tumor burden and poor outcomes, linking increased receptor activation with failure. These limitations resemble the functional defects observed during exhaustion, a cellular state that develops from persistent stimulation of the native T-cell receptor (TCR) in the setting of chronic viral infection, cancer, and autoimmunity. Preclinical studies have confirmed that persistent CAR activation results in a dysfunctional T-cell state that shares many functional attributes of exhaustion. Collectively, these findings indicate that chronic antigen receptor activation is detrimental to both TCR and CAR-driven T cells, and the prevailing presumption is that CAR T cells fail because they become exhausted.

CAR functionality is dependent on activating signals derived from the TCR and a costimulatory receptor. Of the 6 US Food and Drug Agency-approved CAR T-cell products, 2 contain the signaling domain from CD28, the paradigmatic costimulatory with a central role in the endogenous T-cell response. The 4 other products contain the signaling domain from 41BB, a receptor that supports memory T-cell development. Preclinical and clinical data confirm that CAR-integrated costimulatory domains have a significant impact on T-cell function, directing distinct patterns of expansion, persistence, and toxicity. Consideration of CAR costimulatory domains is central to clinical decision making; however, how costimulatory domains contribute to the T-cell dysfunction has not been investigated.

Described herein is an in vitro system to chronically activate CD19−directed CAR T cells with CD19+ acute lymphoblastic leukemia cells. Similar functional defects were observed for both CD28− and 41BB-based CAR T cells but divergent transcriptional, epigenetic, and phenotypic attributes. Although CD28-based CAR T cells bore the hallmarks of T-cell exhaustion, 41BB-based CARs activated a distinct molecular program not previously associated with T-cell dysfunction. Evaluation of CAR T cells collected from a patient at the time of lymphoma progression confirmed expression of this gene signature in an actively failing clinical product. The transcription factor FOXO3 was identified as a driver of 41BB-driven CAR T-cell dysfunction, and disruption of FOXO3 improved function. Together, these data identify a unique CAR-driven dysfunction program activated by 41BB.

### Summary of Methods

Chronic stimulation cultures were established by combining CAR T cells (0.5×10⁶-2×10⁶) with green fluorescent protein-positive Nalm6 cells at an effector-to-target ratio (E:T) of 1:8 in standard media. Cultures were profiled every other day via flow cytometry to both count cells and evaluate changes in protein expression. The cocultures were refed with additional green fluorescent protein-positive Nalm6 cells every other day to maintain an E:T of 1:8 until the onset of dysfunction. CAR T cells were purified on day 0, day 6 or 7 when they were at the peak of activation, and from days 13 to 17 when they became dysfunctional for downstream analyses. Cytometry by time of flight, bulk RNA sequencing (RNAseq), assay for transposase-accessible chromatin with sequencing (ATACseq), and single-cell RNA sequencing (scRNAseq) were performed and analyzed using standard methods. CRISPR editing was performed using the Lonza-4D system with sequencing-based confirmation of gene disruption. Animal studies were performed in nonobese diabetic-severe combined immunodeficiency (NOD-SCID-yc^{-/-}) mice, and disease progression was monitored as previously described.

### Results

### Persistent Exposure to CD19+ Tumors Drives CAR T-Cell Dysfunction

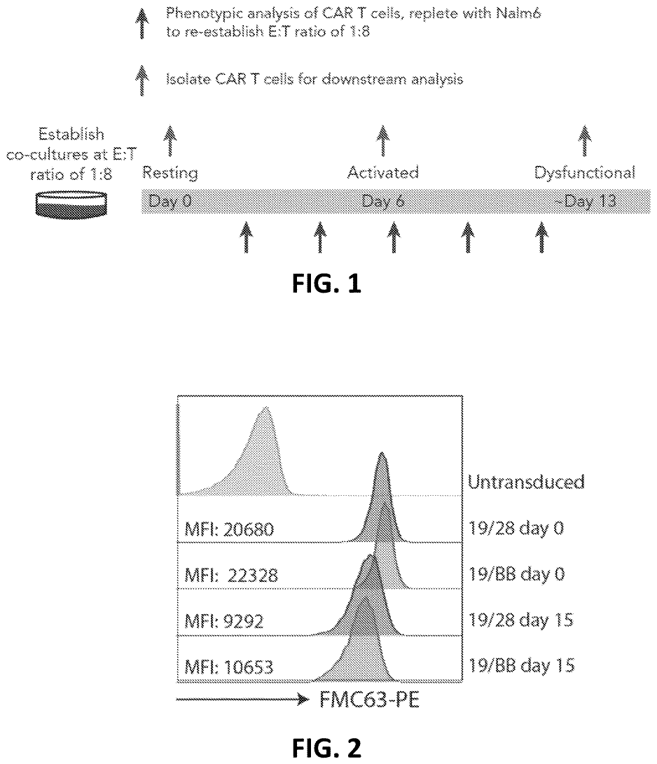

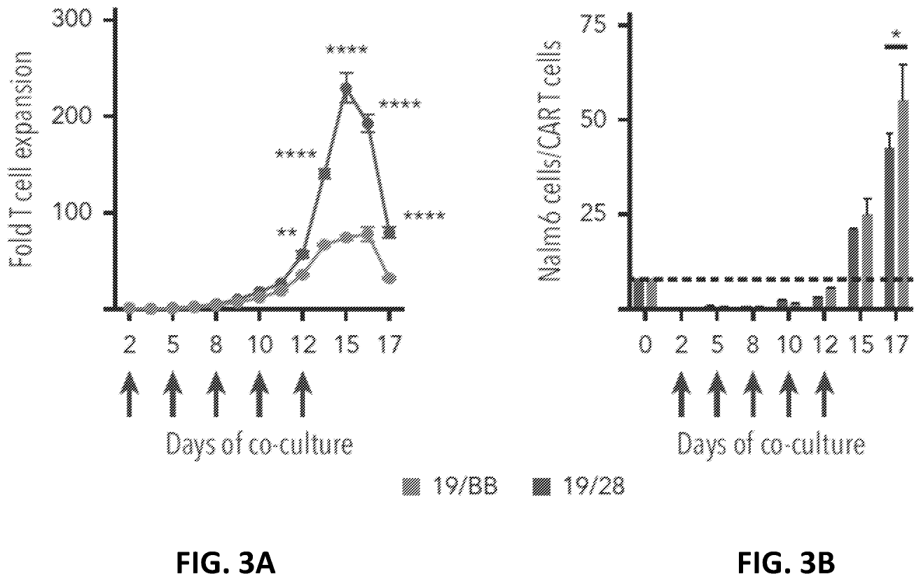

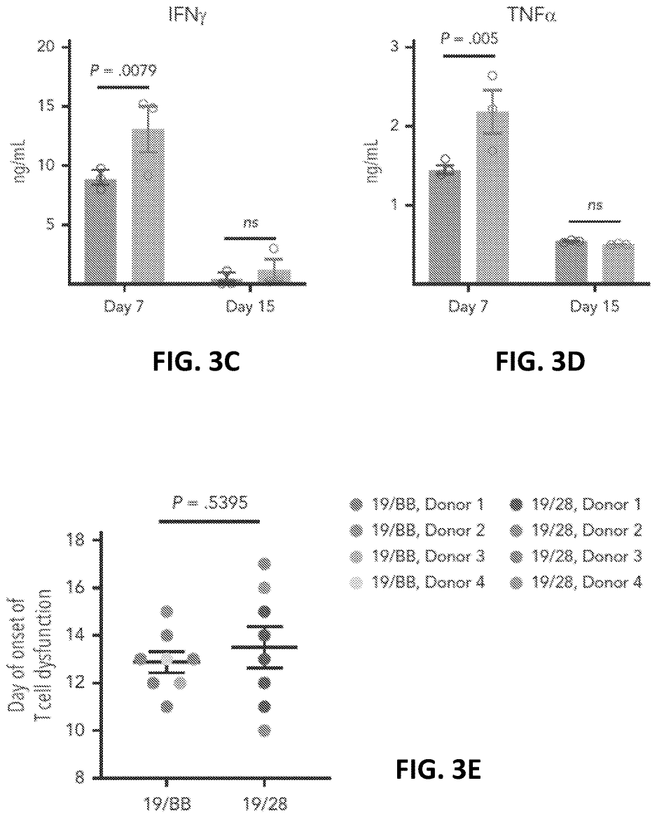

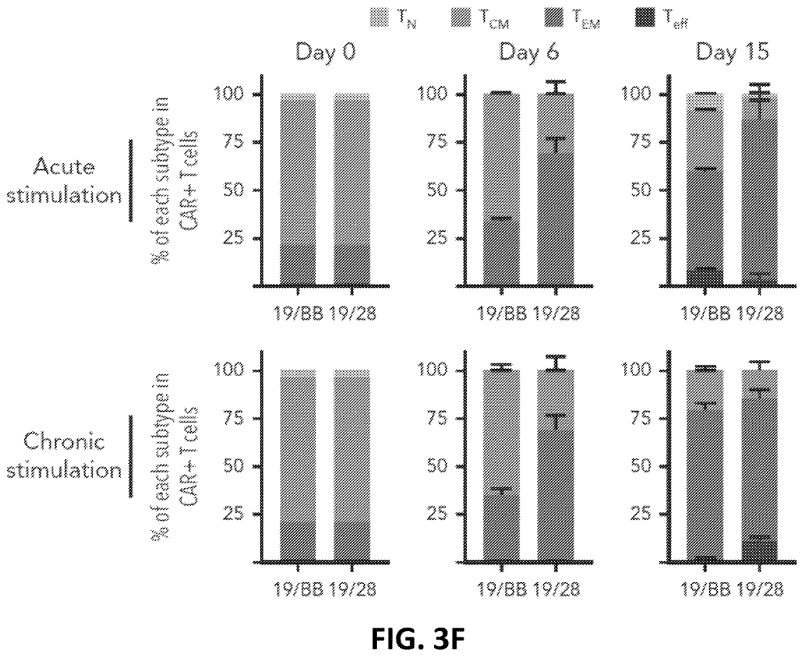

To understand how prolonged costimulatory domain activation affects CAR T-cell function, an in vitro model was developed in which CD19-directed CAR T cells containing either CD28 (19/28) or 41BB (19/BB) domains (purified to ensure that all cells expressed the CAR) were combined with the CD19+ acute lymphoblastic leukemia cell line Nalm6. These cocultures were established at an E:T of 1:8. Because CAR T cells eliminated Nalm6 and proliferated in response to activation, cultures were replenished with additional Nalm6 to reestablish an E:T of 1:8 every ˜48 hours, ensuring chronic CAR stimulation (see e.g., FIG. 1). As previously reported, CAR expression declined modestly during coculture (see e.g., FIG. 2). Longitudinal measurement of T-cell proliferation, a core attribute of function, revealed robust expansion of both cell types with greater expansion of 19/28 cells than that of 19/BB cells (see e.g., FIG. 3A), as has been observed with clinical products. After ˜13 days of chronic stimulation, both 19/28 and 19/BB cell types lost proliferative capacity and contracted, reflecting loss of function. Because cumulative Nalm6 survival was not longitudinally measurable in this refeeding model, the E:T over time was used as a measure of cytotoxic function. Both 19/28 and 19/BB cells demonstrated robust cytotoxicity against persistently high tumor burdens until approximately day 13, at which time both cell types lost the ability to kill (see e.g., FIG. 3B). 19/28 and 19/BB cells were isolated after 7 days of chronic stimulation and restimulated with fresh Nalm6, which elicited potent secretion of interferon γ and tumor necrosis factor α (see e.g., FIG. 3C-FIG. 3D). In contrast, cells isolated after 15 days of chronic stimulation secreted almost no cytokine in response to restimulation. These assays were repeated using T cells derived from 4 independent donors, and although each donor demonstrated distinct kinetics of cytotoxicity, no difference in the timing of dysfunction onset (see e.g., FIG. 3E), defined as either expansion of Nalm6 or CAR T-cell contraction, was found based on CAR costimulatory domain.

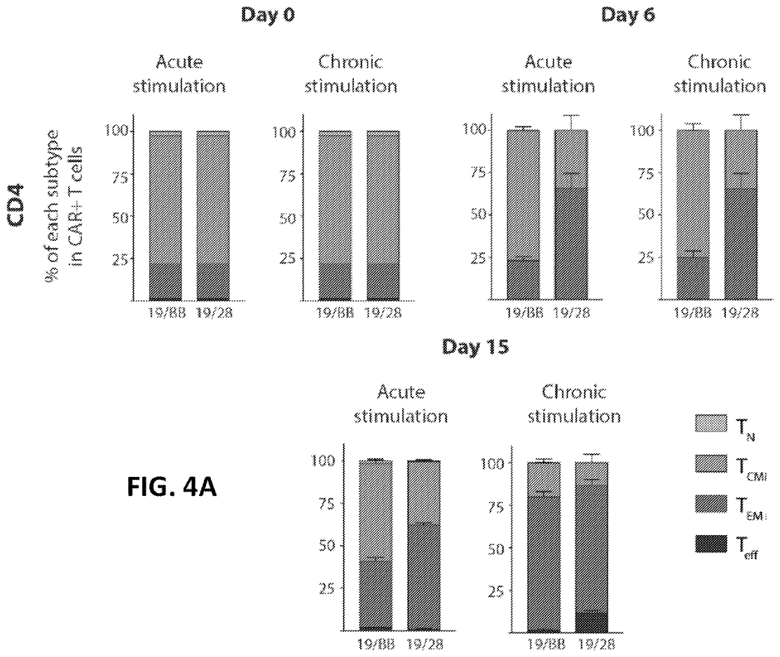

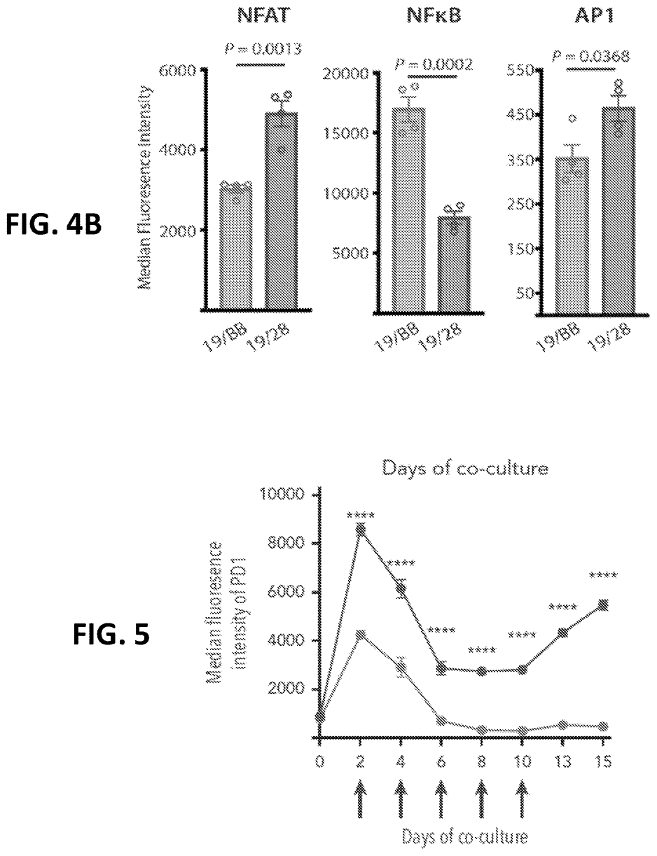

Next, T-cell phenotypes were analyzed over the course of chronic stimulation. CD8 memory differentiation was largely similar for 19/28 and 19/BB cells and it was confirmed that 41BB promotes earlier-lineage memory phenotypes (see e.g., FIG. 3F). This was most pronounced in the setting of acute stimulation, in which cocultures were established but never replenished with Nalm6. A similar trajectory was observed for CD4 T cells (see e.g., FIG. 4A). Next, Jurkat cells engineered to express distinct fluorescent proteins when the transcription factors NFAT, NF-κB, and activator protein 1 (AP1) are activated were used to assess changes in transcriptional regulation. Chronic stimulation of 19/28 Jurkats resulted in increased NFAT and AP1 activity whereas 19/BB Jurkats had higher NF-κB activity (see e.g., FIG. 4B). Persistent activation of NFAT and AP1 reportedly drives exhaustion in models of chronic viral infection and cancer. Next, expression of PD1, the paradigmatic marker of exhaustion, was evaluated. Both 19/28 and 19/BB cells demonstrated an initial increase in PD1 expression, reflecting activation but only 19/28 cells demonstrated resurgent expression with the onset of dysfunction (see e.g., FIG. 5). To interrogate T-cell phenotypes with increased granularity, 39 proteins that define T-cell lineages (see e.g., TABLE 1) were profiled.

TABLE 1
CyTOF panel.

| Target | Tag | Clone | Manufacturer | Catalog # |
|---|---|---|---|---|
| CD45 | 089Y | HI30 | Fluidigm | 3089003B |
| Ki-67 | 115In | 8D5 | Thermo | MA5-15690 |
| CD3 | 141Pr | UCHT1 | Fluidigm | 3141019B |
| CD28 | 142Nd | CD28.2 | biolegand | 302902 |
| CD45Ra | 143Nd | H100 | Fluidigm | 3143006B |
| CD69 | 144Nd | FN50 | Fluidigm | 3144018B |
| CD4 | 145Nd | RPA-T4 | Fluidigm | 3145001B |
| CD8a | 146Nd | RPA-T8 | Fluidigm | 3146001B |
| RORgt | 147Sm | AFKJS-9 | ebioscience | 14-6988-82 |
| CD278/ICOS | 148Nd | C398.4A | Fluidigm | 3148019B |
| CD56 (NCAM) | 149Sm | NCAM16.2 | Fluidigm | 3155008B |
| CD223/LAG-3 | 150Nd | 11C3C65 | Fluidigm | 3150030B |
| CD103 | 151Eu | Ber-ACT8 | Fluidigm | 3151011B |
| TCRgd | 152Sm | 11F2 | Fluidigm | 3152008B |
| CD62L | 153Eu | DREG56 | Fluidigm | 3153004B |
| TIM3 | 154Sm | F38-2E2 | Fluidigm | 3154010B |
| CD27 | 155Gd | L128 | Fluidigm | 3158010B |
| CXCR3 | 156Gd | G025H7 | Fluidigm | 3156004B |
| CD134 (OX40) | 158Gd | ACT35 | Fluidigm | 3158012B |
| CD197 (CCR7) | 159Tb | G043H7 | Fluidigm | 3159003A |
| CD39 | 160Gd | A1 | Fluidigm | 3160004B |
| Tbet | 161Dy | 4B10 | Fluidigm | 3161014B |
| FoxP3 | 162Dy | PCH101 | Fluidigm | 3162011A |
| Eomes | 163Dy | WD1928 | ebioscience | 14-4877-82 |
| CD357 (GITR) | 164Dy | 621 | biolegand | 311602 |
| CD45RO | 165Ho | UCHL1 | Fluidigm | 3149001B |
| CD44 | 166Er | BJ18 | Fluidigm | 3166001B |
| Gata3 | 167Er | TWAJ | Fluidigm | 3167007A |
| CD154 (CD40L) | 168Er | 24-31 | Fluidigm | 3168006B |
| CD25 (IL-2R) | 169Tm | 2A3 | Fluidigm | 3169003B |
| CTLA-4 | 170Er | 14D3 | Fluidigm | 3170005B |
| Granzyme B | 171Yb | GB11 | Fluidigm | 3171002B |
| CD38 | 172Yb | HIT2 | Fluidigm | 3172007B |
| CD137/4-1BB | 173Yb | 4B4-1 | Fluidigm | 3173015B |
| CD279 (PD-1) | 174Yb | EH12.2H7 | Fluidigm | 3174020B |
| Perforin | 175Lu | B-D48 | Fluidigm | 3175004B |
| CD127 | 176Yb | A019D5 | Fluidigm | 3176004B |
| TIGIT | 209Bi | MBSA43 | Fluidigm | 3209013B |

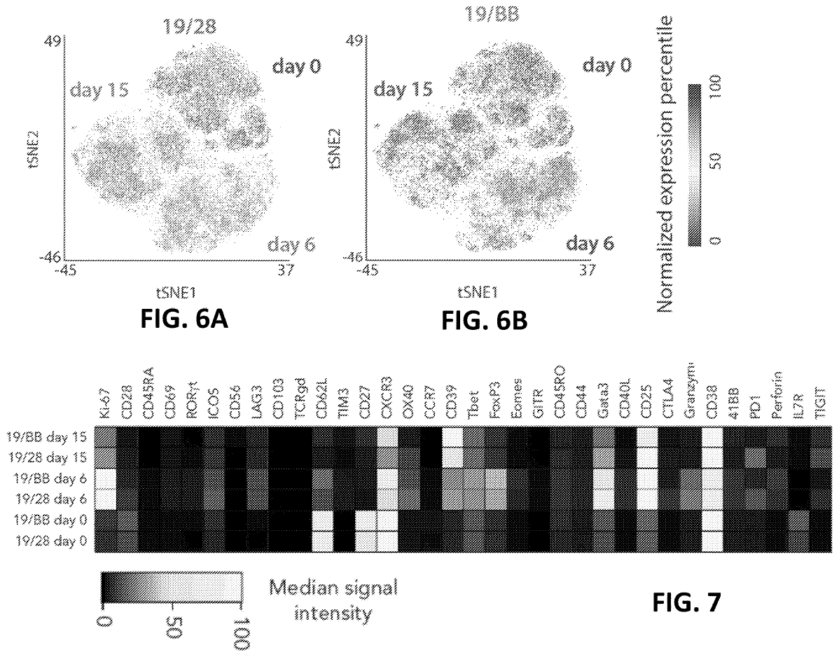

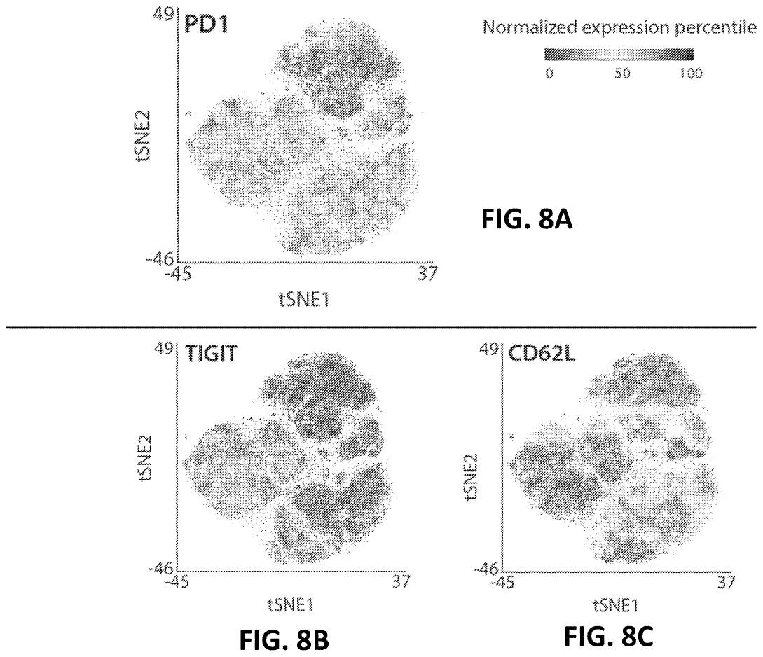

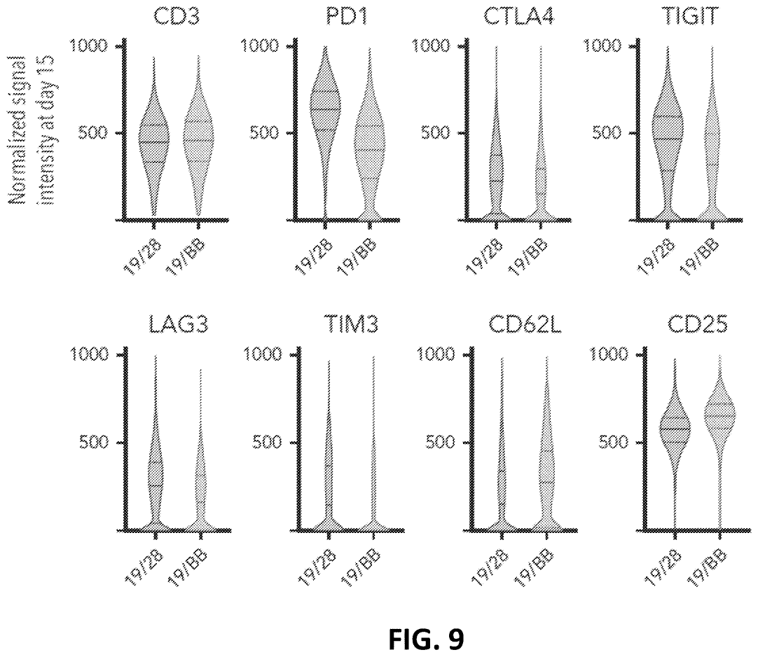

Cytometry by time of flight analysis revealed that prestimulation (day 0) and peak-stimulation (day 6) 19/28 and 19/BB cells had very similar phenotypes, however dysfunctional (day 15) samples occupied different spaces by nearest neighbor (t-distributed stochastic neighbor embedding) analysis (see e.g., FIG. 6A-FIG. 6B). Evaluation of individual markers revealed that day-15 19/28 cells expressed higher levels of PD1 and TIGIT than all other samples, whereas day 19/BB cells expressed higher CD62L (see e.g., FIG. 7 and FIG. 8A-FIG. 8C). Dedicated comparison of the day-15 samples further revealed that 19/28 cells expressed higher levels of exhaustion markers CTLA4, LAG3, and TIM3, whereas 19/BB cells were higher for CD25 (see e.g., FIG. 9), reflecting that dysfunctional 19/28 cells were phenotypically similar to exhausted T cells.

### Dysfunctional CD28− and 41BB-Based CAR T Cells are Programmatically Distinct

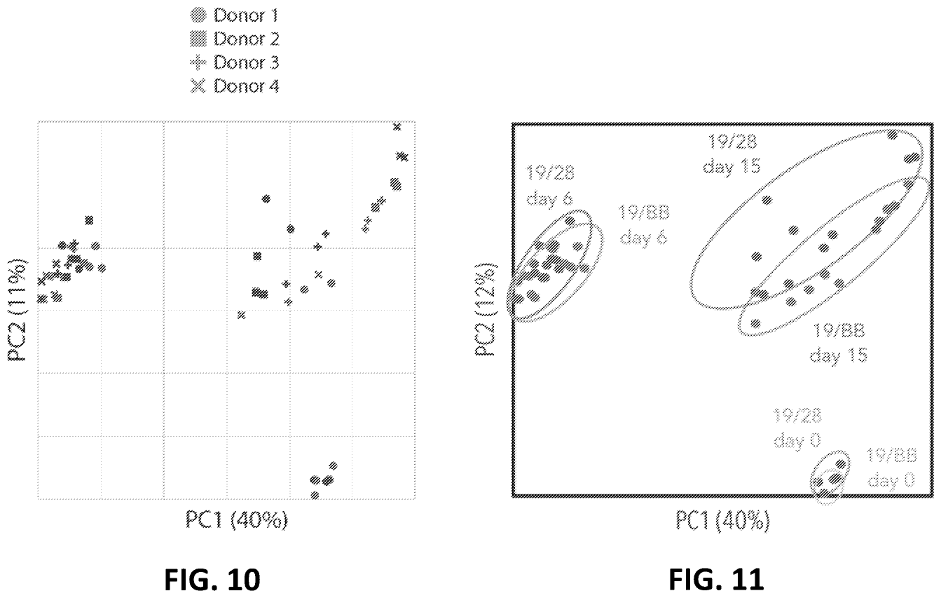

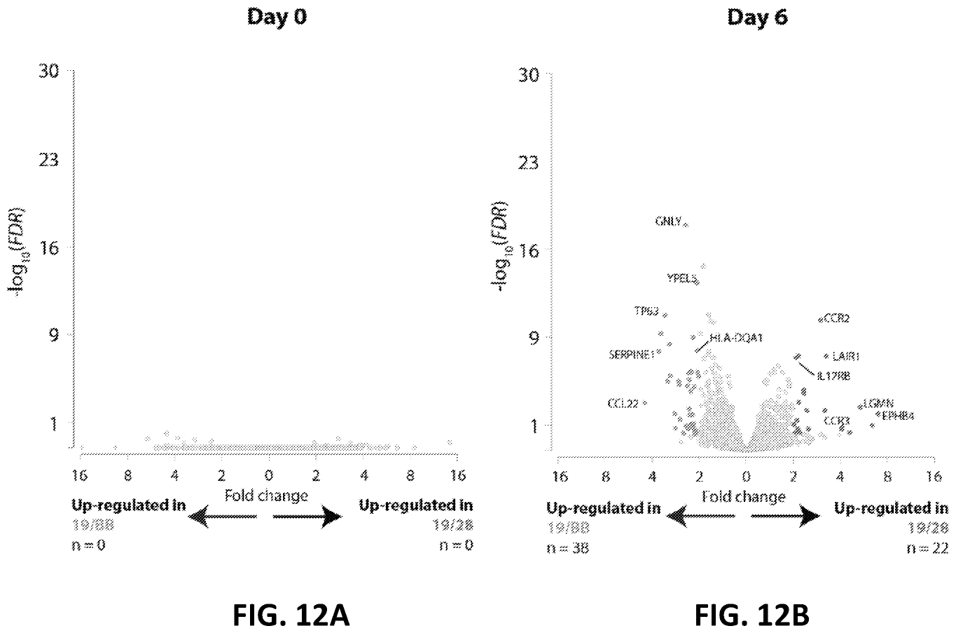

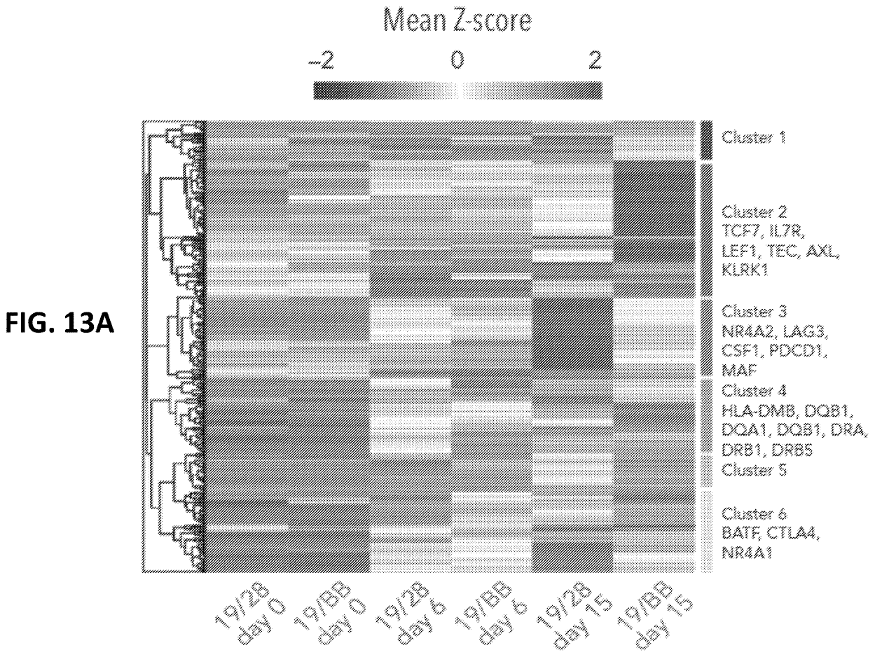

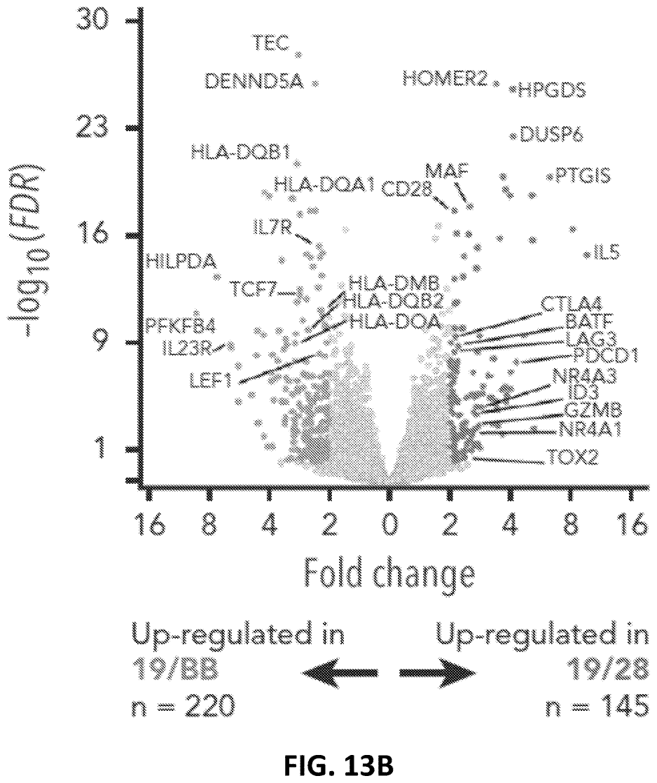

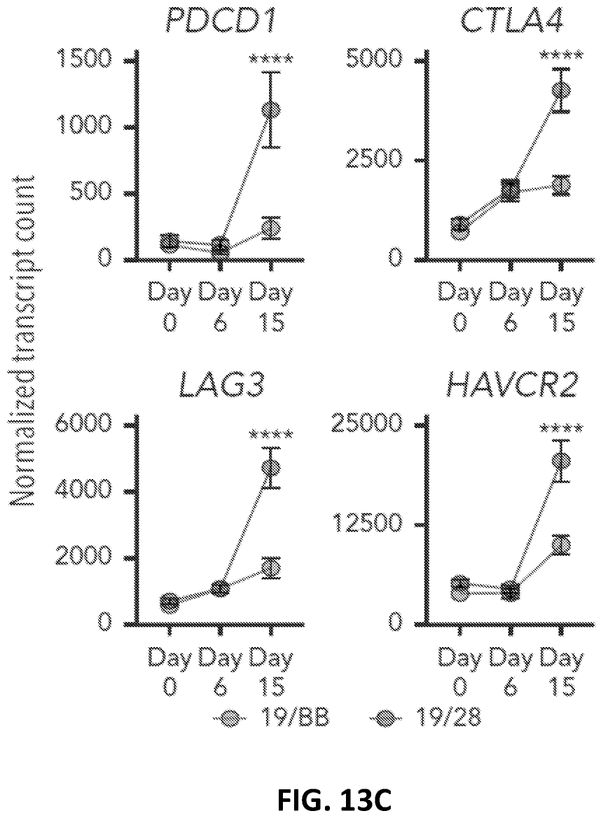

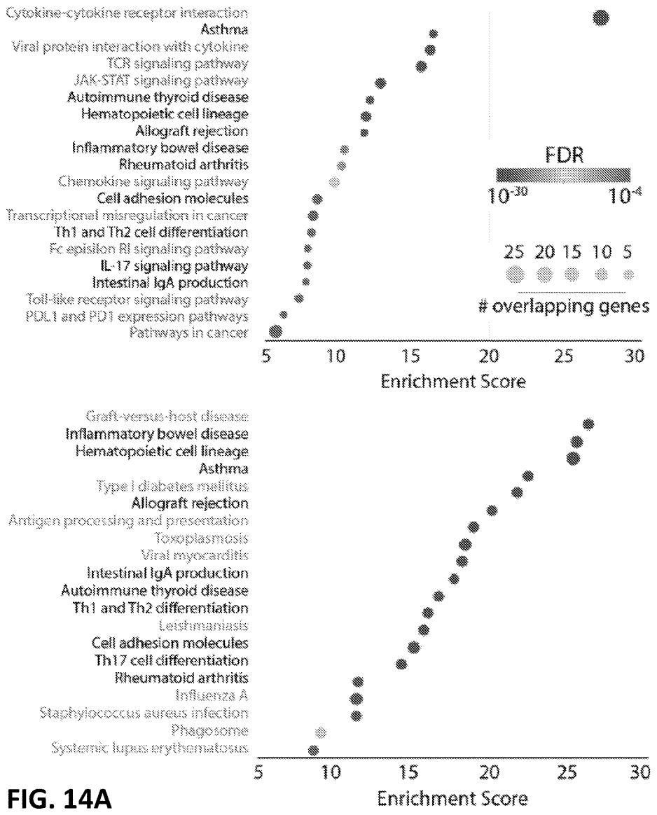

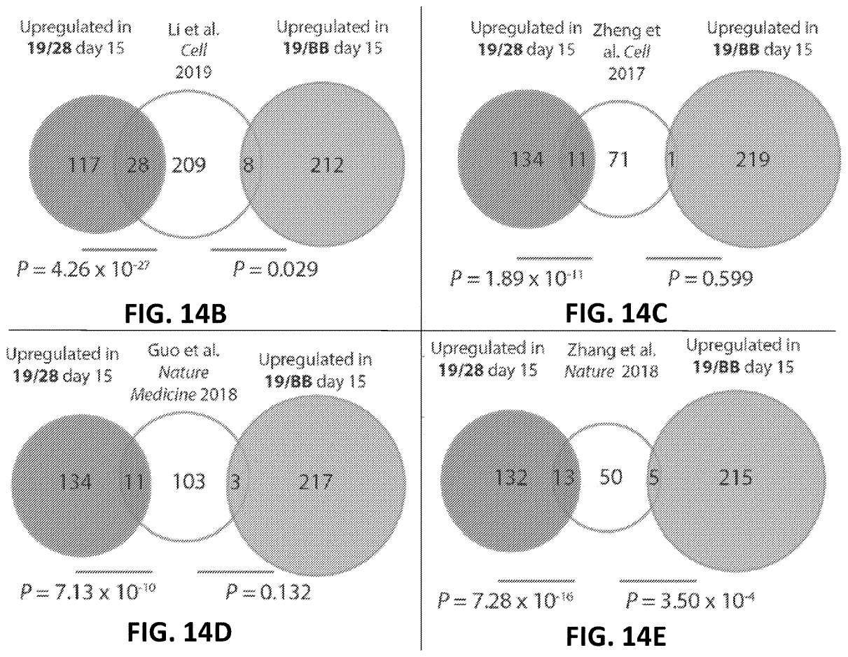

To interrogate the gene expression programs associated with these distinct phenotypic states, RNA sequencing of CAR T cells was performed at days 0, 6, and 15 of chronic stimulation. Principal component analysis demonstrated minimal differences based on donor (see e.g., FIG. 10) or between 19/28 and 19/BB cells at days 0 and 6 but a divergence at day 15 (see e.g., FIG. 11). Although day-6 cells segregated from day 0 cells on both PC1 and PC2, day-15 cells were similar to day-0 cells on PC1, suggesting a regression to a resting-like state. Pooled analysis of samples from all donors demonstrated no differentially expressed genes (DEGs) on day 0 and only 60 DEGs at day 6 (see e.g., FIG. 12A-FIG. 12B). After the onset of dysfunction, however, 365 DEGs were identified that segregated into 6 clusters (see e.g., FIG. 13A). Clusters (3 and 6) with higher expression in dysfunctional 19/28 cells were highly enriched for exhaustion-associated genes, whereas clusters (2 and 4) with higher expression in dysfunctional 19/BB cells were instead enriched for memory markers and class II HLA genes. Visualization by volcano plot underscored the significantly greater expression of exhaustion-associated genes in 19/28 cells (see e.g., FIG. 13B). Tracing expression of exhaustion markers PDCD1, CTLA4, LAG3, and HAVCR2 over time demonstrated a significant rise in expression from day 6 to day 15 in 19/28 cells, as is observed in classical exhaustion, whereas expression of these genes in 19/BB cells only mildly increased over time (see e.g., FIG. 13C). Pathway enrichment analysis of DEGs at day 15 demonstrated some overlap but also enrichment of unique pathways, including TCR signaling, JAK-STAT signaling, and PDL1 and PD1 expression in 19/28 cells, and graft-versus-host disease and antigen processing and presentation in 19/BB cells (see e.g., FIG. 14A).

To quantify the similarity of dysfunctional CAR T-cell states to exhausted human T cells, 4 recent studies that profiled gene expression in exhausted tumor-infiltrating lymphocytes (TILs) were reviewed. Significantly greater overlap was found between dysfunctional 19/28 cells and all human exhaustion gene sets than for dysfunctional 19/BB cells (see e.g., FIG. 141B-FIG. 14E). These TIL gene sets were cross-referenced to identify genes shared by at least 2 gene sets (n=89), and genes shared by all 4 gene sets (master exhaustion gene set, n=17; TABLE 2).

TABLE 2
Human T cell exhaustion genesets.

| Li | Guo Nature | Zheng | Zhang | Present |
|---|---|---|---|---|
| Cell 2019 | Med 2018 | Cell 2017 | Nature 2018 | in >2 |
| ABI3 | ACP5 | ACP5 | ACP5 | ACP5 |
| ADAM28 | ALOX5AP | ADGRG1 | AFAP1L2 | AFAP1L2 |
| AFAP1L2 | ANXA5 | AFAP1L2 | ANXA5 | AKAP5 |
| AKAP5 | APOBEC3C | AKAP5 | APOBEC3C | ANXA5 |
| ANAPC1P1 | ATP6V1C2 | BST2 | APOBEC3G | APOBEC3C |
| ANLN | BHLHE40-AS1 | CCL3 | BATF | BST2 |
| AOAH | BST2 | CCND2 | BST2 | CCL3 |
| APOBEC3B | BTLA | CD27 | CCL3 | CCL4 |
| ASF1B | CCL3 | CD27-AS1 | CCL4 | CCL4L1 |
| ASPM | CCL4 | CD2BP2 | CCL4L1 | CCND2 |
| ATP8B4 | CCND2 | CD38 | CCR1 | CCR1 |
| AURKB | CD200 | CD63 | CD27 | CD200 |
| BIRC5 | CD27 | CHST12 | CD27-AS1 | CD27 |
| BTBD16 | CD27-AS1 | CREM | CD38 | CD27-AS1 |
| BUB1 | CD38 | CSF1 | CD63 | CD38 |
| BUB1B | CD63 | CTLA4 | CD82 | CD63 |
| C16orf45 | CD7 | CTSD | COX5A | CD82 |
| C2orf48 | CD82 | CXCL13 | CTLA4 | COX5A |
| CADM1 | CDK2AP2 | CXCR6 | CTSD | CSF1 |
| CCL3 | CKS2 | DFNB31 | CTSW | CTLA4 |
| CCL3L1 | COTL1 | DUSP4 | CXCL13 | CTSD |
| CCL3L3 | COX5A | ENTPD1 | CXCR6 | CTSW |
| CCL4 | CREG1 | ENTPD1-AS1 | DUSP4 | CXCL13 |
| CCL4L1 | CTLA4 | FKBP1A | ENTPD1 | CXCR6 |
| CCL5 | CTSD | FUT8 | FASLG | DUSP4 |
| CCNB1 | CXCL13 | GALM | FKBP1A | ENTPD1 |
| CCNB2 | CXCR6 | GZMB | FKBP1A-SDCBP2 | FASLG |
| CCR1 | DDIT4 | HAVCR2 | FUT8 | FKBP1A |
| CD200 | DNPH1 | HLA-DMA | GSTO1 | FKBP1A-SDCBP2 |
| CD244 | DTHD1 | HLA-DRA | GZMB | FUT8 |
| CD248 | DUSP4 | HMGN3 | GZMH | GALM |
| CD38 | DYNLL1 | ICOS | HAVCR2 | GZMB |
| CD63 | ENTPD1 | ID3 | HLA-DQA1 | GZMH |
| CD8A | FABP5 | IFI35 | HLA-DQB1 | HAVCR2 |
| CD8B | FASLG | IFNG | HLA-DRA | HLA-DMA |
| CD9 | FKBP1A | IGFLR1 | HLA-DRB1 | HLA-DQA1 |
| CDC20 | FKBP1A-SDCBP2 | IL2RB | HLA-DRB5 | HLA-DRA |
| CDC45 | FKBP5 | ITGAE | HLA-DRB6 | HLA-DRB1 |
| CDC6 | GALM | ITM2A | IFI27L2 | HLA-DRB5 |
| CDCA2 | GPR25 | LAG3 | IFI6 | HLA-DRB6 |
| CDCA3 | GZMB | LAYN | IFNG | ID3 |
| CDCA5 | HAVCR2 | LINC00299 | IGFLR1 | IFI35 |
| CDCA8 | HLA-DRA | LYST | ISG15 | IFNG |
| CDK1 | HLA-DRB5 | MIR155HG | ITGAE | IGFLR1 |
| CDKN3 | HLA-DRB6 | MIR4632 | ITM2A | ISG15 |
| CDT1 | HMGN1 | MS4A6A | LAG3 | ITGAE |
| CENPA | ID2 | MTHFD1 | MIR155 | ITM2A |
| CENPW | IDH2 | MTHFD2 | MIR155HG | KRT81 |
| CEP55 | IFI35 | MYO1E | MIR497HG | KRT86 |
| CKAP2L | IFNG | MYO7A | MYO7A | LAG3 |
| CLEC12A | IGFLR1 | NAB1 | NAB1 | LAYN |
| CLIC3 | IL21 | NDFIP2 | NDFIP2 | LINC00299 |
| CLNK | IL6ST | PARK7 | PARK7 | MIR155 |
| CLSPN | ISG15 | PDCD1 | PDCD1 | MIR155HG |
| CRTAM | ITGAE | PHLDA1 | PHLDA1 | MIR4632 |
| CSF1 | ITM2A | PKM | PKM | MIR497HG |
| CST7 | ITPR1 | PRDM1 | PTTG1 | MTHFD2 |
| CTSW | KRT81 | PRDX5 | RBPJ | MYO1E |
| CXCL13 | KRT86 | PRF1 | SIRPG | MYO7A |
| CXXC5 | LAG3 | PRKAR1A | SIT1 | NAB1 |
| DBN1 | LAYN | RAB27A | SNAP47 | NDFIP2 |
| DEPDC1B | LIMS1 | RALGDS | TIGIT | PARK7 |
| DIAPH3 | LINC00892 | RGS1 | TNFRSF18 | PDCD1 |
| DLGAP5 | LRMP | RGS2 | TNFRSF9 | PHLDA1 |
| DMC1 | MIR155 | SARDH | TNFSF4 | PKM |
| DRAXIN | MIR155HG | SIRPG | TNIP3 | PRDX5 |
| DSCC1 | MIR3917 | SNAP47 | TOX | PRF1 |
| DTL | MIR4632 | SNX9 | VCAM1 | PTTG1 |
| E2F8 | MIR497HG | STAT3 | | RAB27A |
| EGR2 | MTHFD2 | SYNGR2 | | RBPJ |
| ENTPD1 | MYO7A | TIGIT | | RGS1 |
| EOMES | NAP1L4 | TNFRSF1B | | RGS2 |
| ESCO2 | NDFIP2 | TNFRSF9 | | SARDH |
| ETV1 | NR3C1 | TOX | | SIRPG |
| EXO1 | PARK7 | TPI1 | | SIT1 |
| FAM111B | PDCD1 | TRAFD1 | | SNAP47 |
| FAM166B | PDIA6 | UBE2F | | SNX9 |
| FAM3C | PHLDA1 | UBE2F-SCLY | | STMN1 |
| FAM49A | PRDX3 | VAPA | | SYNGR2 |
| FASLG | PRDX5 | VCAM1 | | TIGIT |
| FBXO43 | PSMB3 | WARS | | TNFRSF18 |
| FCGR3A | PSMC3 | YARS | | TNFRSF9 |
| FCRL3 | PSMD4 | | | TNFSF4 |
| FXYD2 | PSMD8 | | | TNIP3 |
| FXYD6-FXYD2 | PTPN13 | | | TOX |
| GABARAPL1 | PTTG1 | | | TPI1 |
| GCNT1 | RAB27A | | | VCAM1 |
| GEM | RANBP1 | | | ZBED2 |
| GFOD1 | RBPJ | | | |
| GGH | RGS1 | | | |
| GINS1 | RGS2 | | | |
| GINS2 | RNF19A | | | |
| GNG4 | SAMSN1 | | | |
| GNGT2 | SARDH | | | |
| GNLY | SIRPG | | | |
| GOLIM4 | SIT1 | | | |
| GPA33 | SMCO4 | | | |
| GTSE1 | SNAP47 | | | |
| GZMA | SNRPB | | | |
| GZMB | SNX9 | | | |
| GZMH | SRGN | | | |
| HAVCR2 | STMN1 | | | |
| HIST1H1B | STRA13 | | | |
| HIST1H2AL | SYNGR2 | | | |
| HJURP | TIGIT | | | |
| HLA-DMA | TMEM243 | | | |
| HLA-DQA1 | TNFRSF18 | | | |
| HLA-DQA2 | TNFRSF1B | | | |
| HLA-DRB1 | TNFRSF9 | | | |
| HLA-DRB6 | TNFSF8 | | | |
| HMMR | TNIP3 | | | |
| HMOX1 | TOX | | | |
| ID3 | TPI1 | | | |
| IFITM10 | UBE2L6 | | | |
| IFNG | VCAM1 | | | |
| IGFBP6 | YWHAQ | | | |
| IRF8 | ZBED2 | | | |
| ITGA1 | | | | |
| ITGA2 | | Present in all 4 | | |
| ITGAE | | CCL3 | | |
| ITM2C | | CSF1 | | |
| KIF14 | | CTLA4 | | |
| KIF15 | | CXCL13 | | |
| KIF18B | | CXCR6 | | |
| KIF23 | | HAVRC2 | | |
| KIF2C | | IFNG | | |
| KIF4A | | ITGAE | | |
| KIR2DL3 | | LAG3 | | |
| KIR2DL4 | | LYST | | |
| KLRC1 | | MYO7A | | |
| KLRC2 | | PDCD1 | | |
| KLRC3 | | PHLDA1 | | |
| KLRC4 | | RAB27A | | |
| KLRC4-KLRK1 | | SNAP47 | | |
| KLRD1 | | TNFRSF1B | | |
| KLRK1 | | TNFRSF9 | | |
| KRT81 | | | | |
| KRT86 | | | | |
| LAG3 | | | | |
| LAYN | | | | |
| LILRB1 | | | | |
| LILRP2 | | | | |
| LIMK1 | | | | |
| LINC00158 | | | | |
| LINC00299 | | | | |
| LRRC28 | | | | |
| MCM10 | | | | |
| MCM2 | | | | |
| MCM4 | | | | |
| MELK | | | | |
| MKI67 | | | | |
| MND1 | | | | |
| MTFR2 | | | | |
| MYBL2 | | | | |
| MYO1E | | | | |
| MYO7A | | | | |
| NCAPG | | | | |
| NCR1 | | | | |
| NEIL3 | | | | |
| NEK2 | | | | |
| NHS | | | | |
| NKG7 | | | | |
| NUF2 | | | | |
| ORC1 | | | | |
| ORC6 | | | | |
| P2RY1 | | | | |
| PBK | | | | |
| PDLIM4 | | | | |
| PHEX | | | | |
| PHGDH | | | | |
| PHLDA1 | | | | |
| PIK3AP1 | | | | |
| PIK3R6 | | | | |
| PKMYT1 | | | | |
| PLCG2 | | | | |
| PLK1 | | | | |
| POC1A | | | | |
| POLQ | | | | |
| PON2 | | | | |
| PON3 | | | | |
| PRF1 | | | | |
| PROX2 | | | | |
| PRR11 | | | | |
| PRSS30P | | | | |
| PTGIS | | | | |
| PTMS | | | | |
| PTPRK | | | | |
| QPRT | | | | |
| RAD51 | | | | |
| RAD51AP1 | | | | |
| RDH10 | | | | |
| REEP2 | | | | |
| REG4 | | | | |
| RGS13 | | | | |
| RRM2 | | | | |
| SEMA4A | | | | |
| SFTPB | | | | |
| SHCBP1 | | | | |
| SKA1 | | | | |
| SKA3 | | | | |
| SLAMF7 | | | | |
| SLC17A9 | | | | |
| SLC2A8 | | | | |
| SLC43A3 | | | | |
| SMIM10 | | | | |
| SPAG5 | | | | |
| SPC24 | | | | |
| SPC25 | | | | |
| SPRY1 | | | | |
| SPRY2 | | | | |
| STMN1 | | | | |
| STYK1 | | | | |
| SYNGR1 | | | | |
| TIGIT | | | | |
| TIMD4 | | | | |
| TK1 | | | | |
| TMEM155 | | | | |
| TNFRSF9 | | | | |
| TNFSF4 | | | | |
| TNFSF9 | | | | |
| TNIP3 | | | | |
| TNS3 | | | | |
| TOP2A | | | | |
| TPX2 | | | | |
| TRIP13 | | | | |
| TROAP | | | | |
| TSPAN13 | | | | |
| TTC24 | | | | |
| TTK | | | | |
| TYMS | | | | |
| UBE2C | | | | |
| UBE2T | | | | |
| UHRF1 | | | | |
| VCAM1 | | | | |
| WDHD1 | | | | |
| WIPF3 | | | | |
| XCL2 | | | | |
| XRCC2 | | | | |
| YBX3 | | | | |
| ZBED2 | | | | |
| ZBTB32 | | | | |
| ZC3H12C | | | | |
| ZNF683 | | | | |
| ZWINT | | | | |

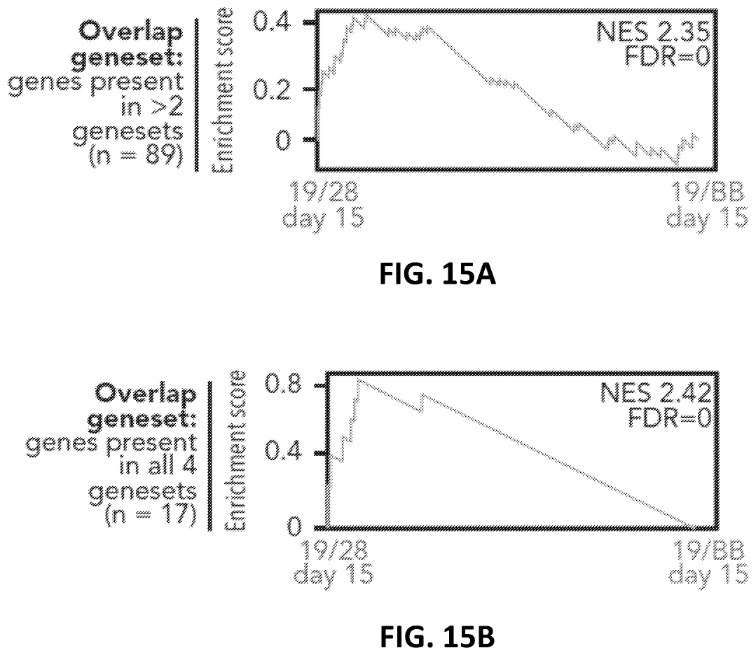

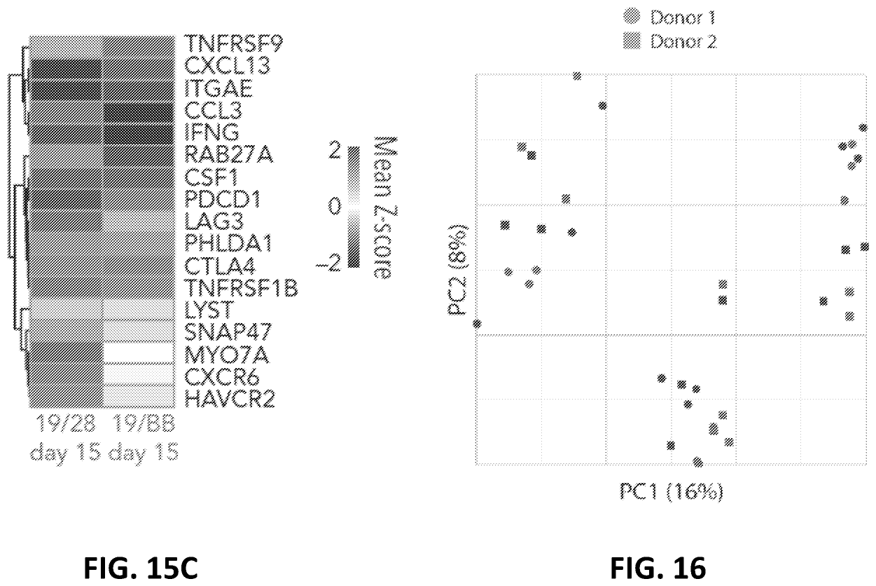

Gene set enrichment analysis demonstrated significant enrichment of both shared gene sets in dysfunctional 19/28 cells as compared with dysfunctional 19/BB cells (see e.g., FIG. 15A-FIG. 15B) and higher overall expression of nearly all 17 master exhaustion genes in dysfunctional 19/28 cells (see e.g., FIG. 15C).

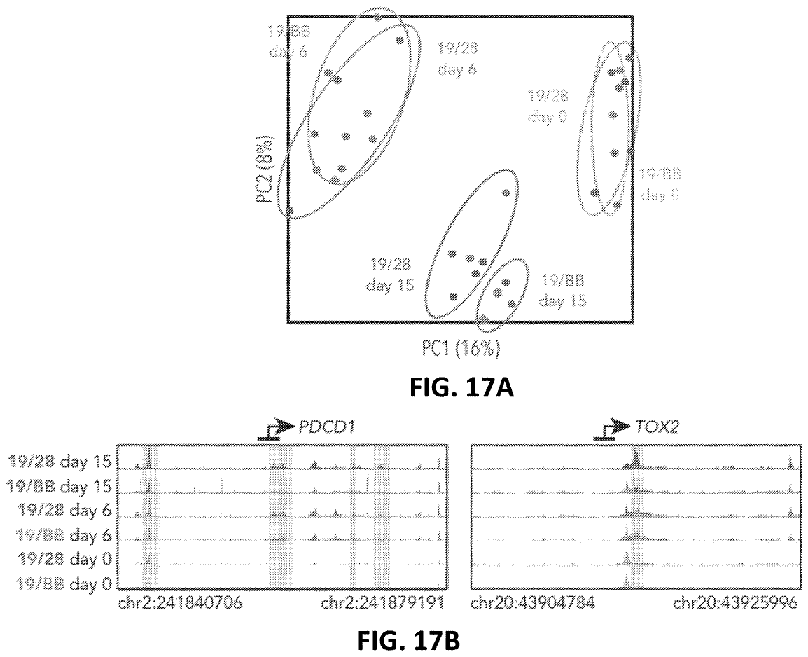

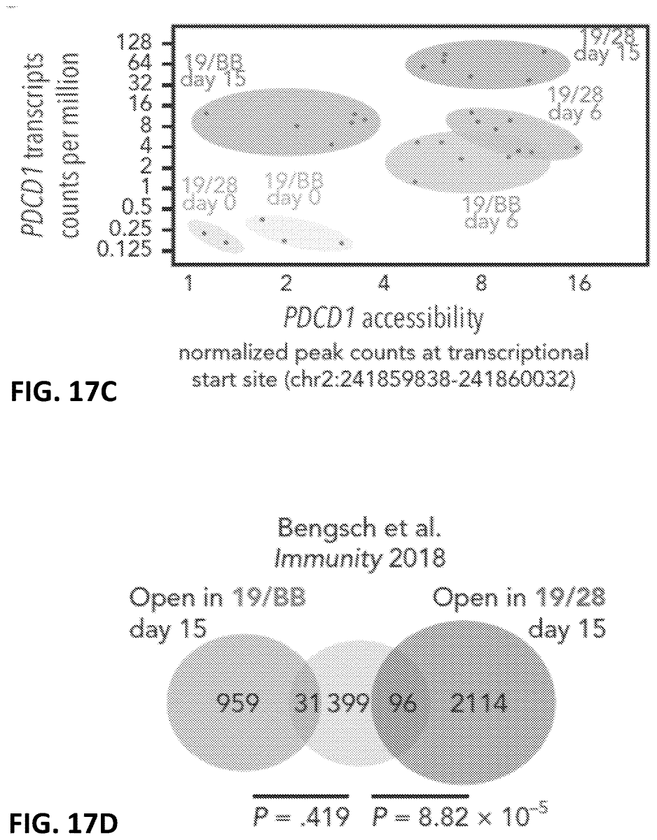

Several studies have demonstrated recurrent epigenetic alterations that define T-cell exhaustion. To probe how chromatin accessibility evolved over the course of chronic CAR stimulation, longitudinal assay for transposase-accessible chromatin was performed with sequencing. Consistent with the transcriptional data, no difference was found based on donor (see e.g., FIG. 16) but a divergence in chromatin accessibility was observed only after the onset of dysfunction at day 15 and, again, it was observed that day-15 cells drifted back toward day-0 cells on PC1 (see e.g., FIG. 17A). Interrogation of the exhaustion-associated genes PDCD1 and TOX2 revealed significantly greater accessibility at transcriptional start sites in dysfunctional 19/28 cells as compared with all other groups (see e.g., FIG. 17B). PDCD1, a gene whose regulation is a defining feature of exhaustion, was focused on and chromatin accessibility and transcript counts to profile gene regulatory dynamics were traced at this site. As in exhausted T cells, 19/28 cells increased PDCD1 expression over time and preserved chromatin accessibility as they progressed from activated to dysfunctional. In contrast, 19/BB cells closed this site to prestimulation levels as they progressed from activated to dysfunctional (see e.g., FIG. 17C), suggesting divergent regulation at this site. Finally, the similarity of these 2 cell states to a previously defined exhaustion-associated chromatin signature was quantitated and, again, significantly greater overlap was found between open chromatin sites in classically exhausted cells and dysfunctional 19/28 cells (P=8.82×10⁻⁵) and minimal overlap with dysfunctional 19/BB cells (P=0.419; see e.g., FIG. 17D). Collectively, these data indicate that dysfunctional 19/28 cells and exhausted T cells share similar transcriptional and epigenetic identities whereas dysfunctional 19/BB cells are programmatically distinct.

### Chronic Stimulation of 19/BB Cells Promotes Development of a Unique Dysfunctional Cell State

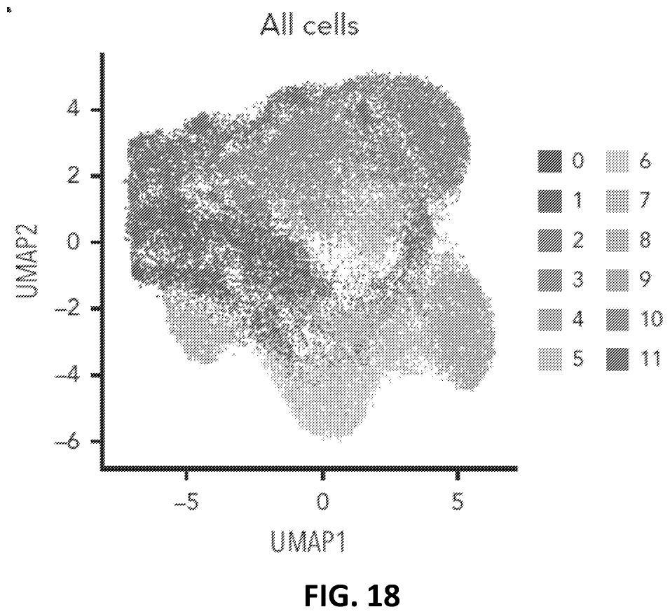

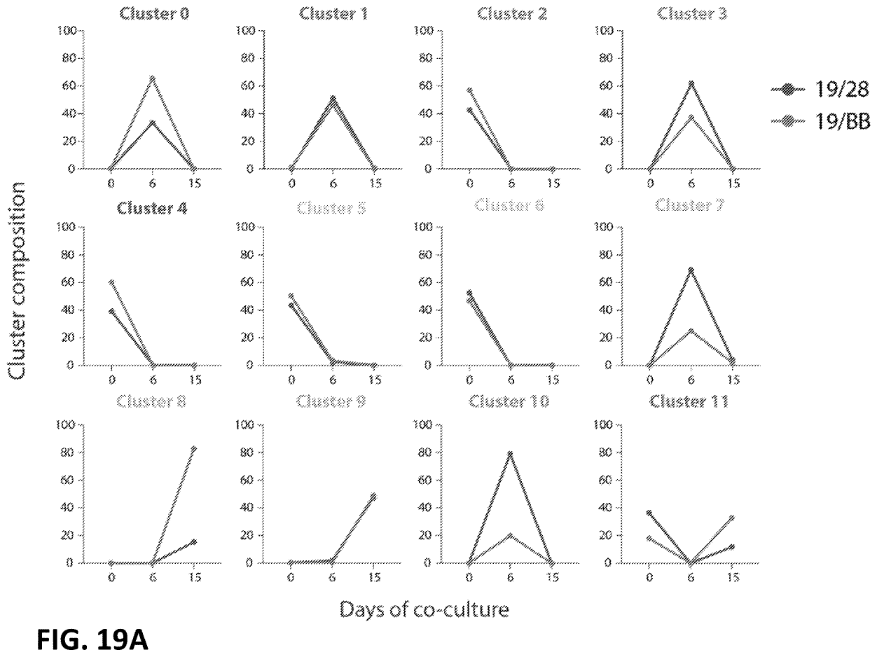

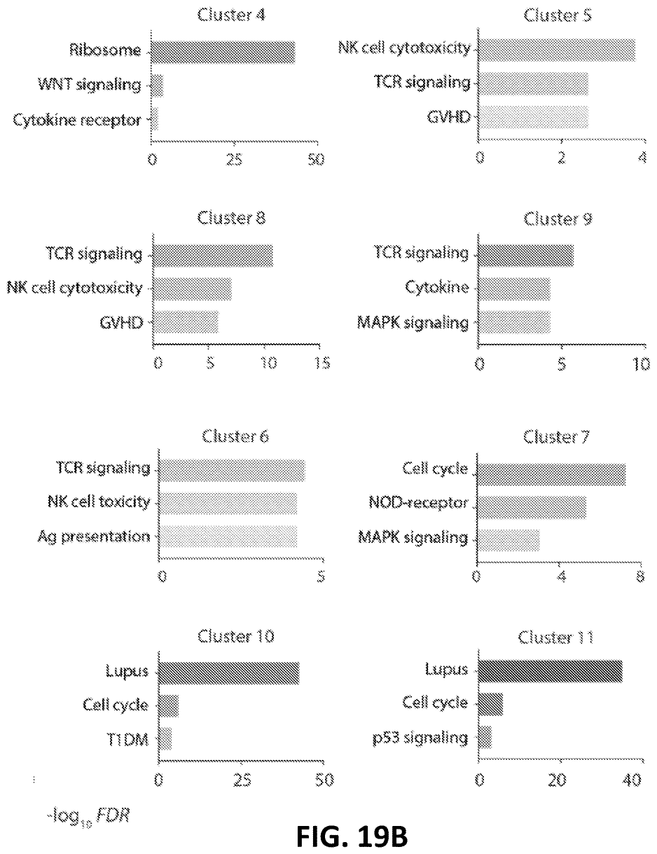

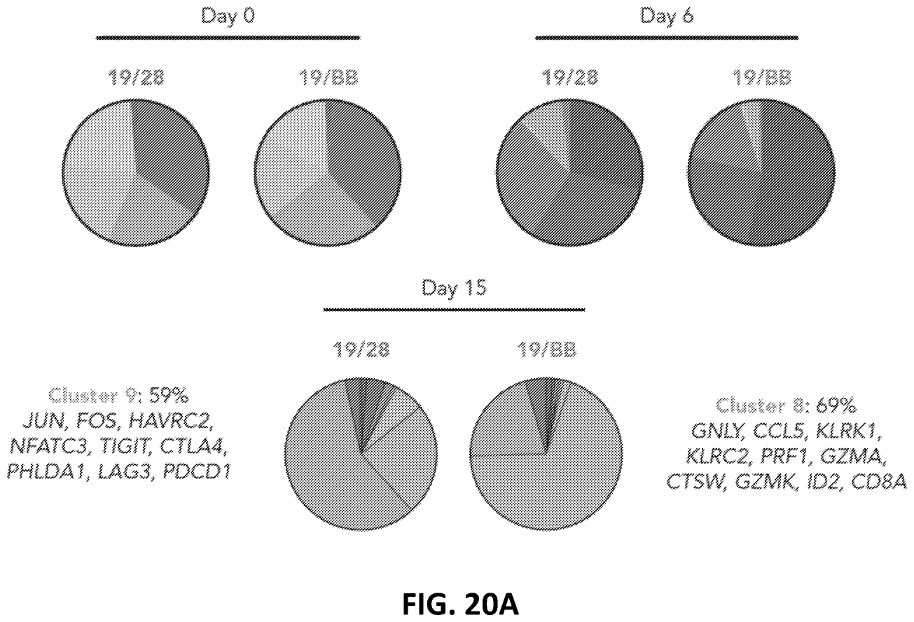

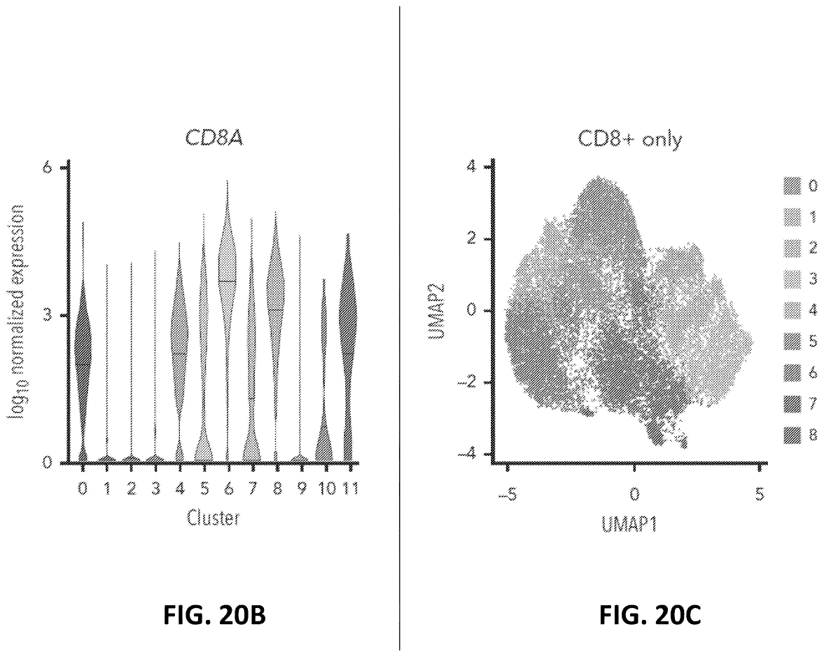

To clarify the distinct cell states that made up the dysfunctional populations, longitudinal scRNAseq was performed. Cells collected at days 0, 6, and 15 segregated into 12 clusters and the clusters (see e.g., FIG. 18) were largely defined by the collection time point (see e.g., FIG. 19A). Pathway analysis confirmed representation of predictable biological programs in each cluster (e.g., cell cycle and metabolism genes in activated clusters 0, 1, 7, and 10, FIG. 19B). The analysis focused on clusters 8 and 9, which were almost entirely composed of dysfunctional cells (cluster 8, 85% day-15 cells; cluster 9, 88% day-15 cells). Notably, no other cluster comprised >5% day-15 cells. Although cluster 9 contained roughly equivalent quantities of cells from both day-15 samples (44% 19/1B cells; 42% 19/28 cells), cluster 8 was heavily skewed toward day-15 19/BB cells (83% 19/BB cells; 17% 19/28 cells; FIG. 19A). Evaluation of each sample individually again demonstrated largely equivalent cluster distributions at days 0 and 6 but distinct cluster distribution at day 15 (see e.g., FIG. 20A). Of dysfunctional 19/28 cells, 59% occupied cluster 9, which was defined by expression of exhaustion-associated genes. In contrast, 69% of dysfunctional 19/BB cells were in cluster 8, whose identity was defined by a distinct set of genes with varied roles in lymphocyte function including cytotoxicity (GNLY, CCL5, PRF1, GZMA, GZMK, and CTSW), natural killer (NK) cell identity (KLRK1 and KLRC2), and T-cell differentiation (ID2). The expression of NK markers has previously been reported in classically exhausted T cells and dysfunctional CAR T cells. Consistent with the modest increase in expression of exhaustion genes (see e.g., FIG. 13C), a fraction of dysfunctional 19/BB cells occupied cluster 9, suggesting that dysfunctional 19/BB cells are heterogeneous and contain some classically exhausted cells and that most have a novel transcriptional identity.

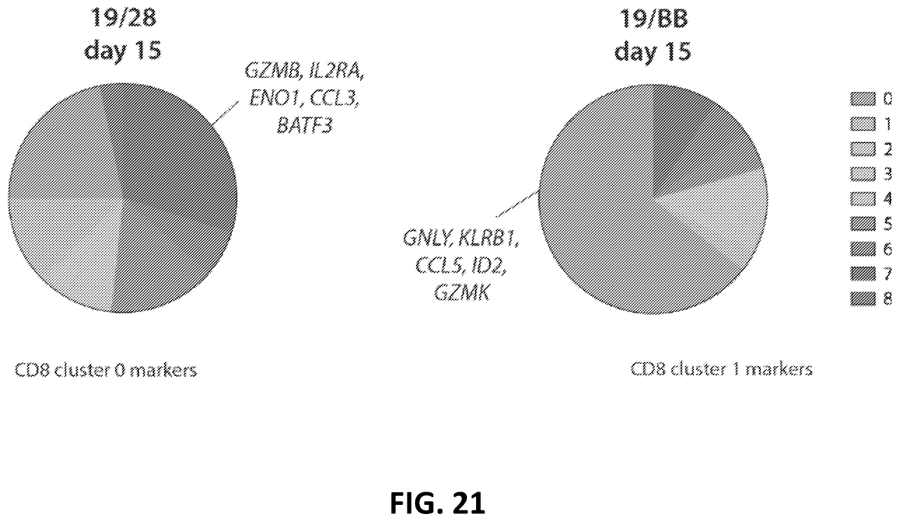

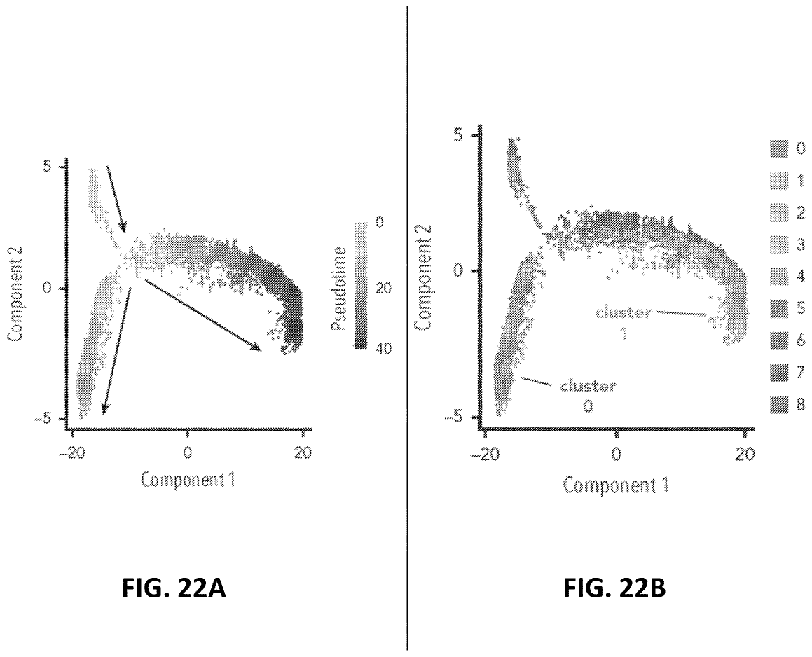

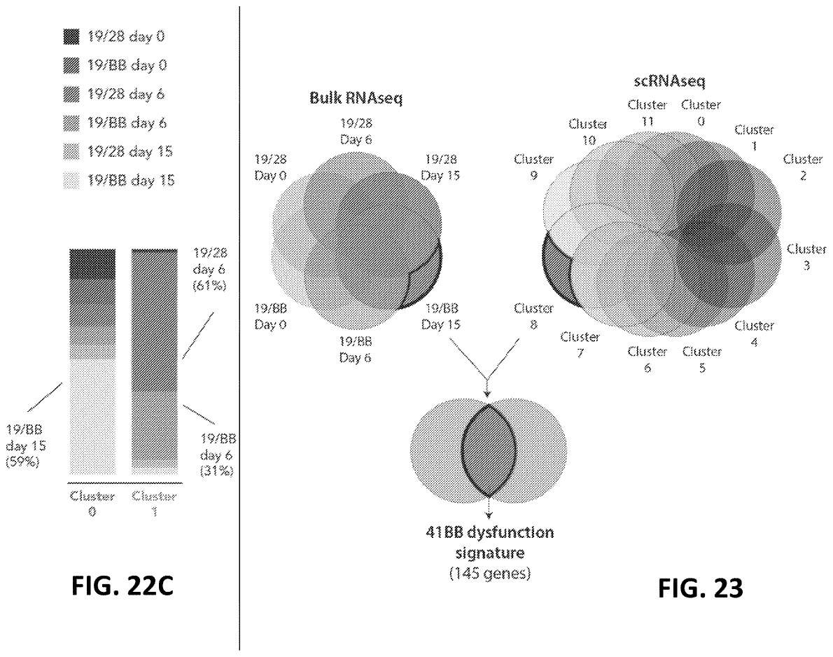

Because cluster 8 cells expressed uniformly high levels of CD8A (see e.g., FIG. 20B), only CD8 cells were reclustered from all 6 samples. CD8 cells distributed to 9 new clusters (see e.g., FIG. 20C), and dysfunctional 19/28 cells primarily occupied CD8-cluster 7, defined by expression of GZMB, IL2RA, ENO1, CCL3, and BATF3 (see e.g., FIG. 21). Dysfunctional 19/BB cells were highly enriched for CD8-cluster 0, defined by expression of GNLY, KLRB1, CCL5, ID2, and GZMK, reflecting similarity in identity of CD8-cluster 0 to cluster 8 from the whole population analysis. To trace the transcriptional evolution of CD8+ CAR T cells, pseudotime analysis was performed. This demonstrated a divergence along 2 developmental trajectories (see e.g., FIG. 22A). Mapping of the CD8 clusters onto pseudotime revealed 2 clusters at the termination of these trajectories: CD8-cluster 0 and CD8-cluster 1 (see e.g., FIG. 22B). CD8-cluster 0 was predominantly composed of day-15 19/BB cells (59%, see e.g., FIG. 22C) and a small fraction of day-15 19/28 cells (6.8%). CD8-cluster 1 was defined by expression of activation markers MKI67, BHLHE40, and TOP2A and, consistently, was almost entirely composed of cells from day 6 (92%; FIG. 22C). These findings suggest that dysfunctional 19/BB cells acquire a terminal T-cell identity.

CAR T Cells that Fail Clinically Acquire a Dysfunctional 19/BB Gene Signature

Using the bulk RNAseq and scRNAseq data sets, a gene signature of dysfunctional 19/BB cells was generated (T_{BBD}; FIG. 23; TABLE 3).

TABLE 3
Dysfunctional 41BB-based CAR T cell (Tbbd) gene signature of dysfunction.
Genes

ABCG1 ADAM8 AHI1 AKNA ALOX5AP ANKRD12 ANKRD28 AOAH ARHGAP9 ARHGEF12
ASB2 ATP8B4 BCL2L11 CAMK4 CBLB CCL5 CCR8 CD2 CD27 CD37 CD3D CD52 CD63 CD69
CD8A CD8B CD96 CDKN1B CFLAR CLEC2B CLEC2D CLSTN3 CST7 CTSW CXCR6CYTH4
DAPK2 DDHD1 DDX17 DOCK8 DUSP1 ELF1 ENTPD1 ERN1 EVA1B EVI2B EVL FASLG
FCER1G FYB1 GABARAPL1 GADD45B GCNT1 GLIPR1 GNLY GOLGA8A GOLGA8B GPR171
GPR174 GZMK HAVCR2 HCST HOPX HSH2D ID2 IER5L IGFLR1 IKZF2 IL10RA IL16 IL2RG
IL9R INPP5D ITGA1
ITGAX JAK3 JAML JUN KDM5B KIT KLF6 KLRB1 KLRC2 KLRC3 KLRC4 KLRD1 KLRK1 KMT2E
LNPEP LRRC28 LYST MCL1 MCTP2 MEF2A NBL1
NCAM1 NCF4 NCR3 NELL2 NFATC3 NLRC3 NT5E OGT PAG1 PDCD4 PDE3B PDE7A PDE7B
PGGHG PLAAT4 PPP1R15A PRF1 PRKCA PTGER4 PTPN22 RAB24 RAB37 RASGRP1
RBM39 RBPJ REG4 RESF1 RIN3 RPS6KA3 SCLY SERPINE1 SIRPG SKAP1 SLAMF7 SLC1A4
SMAD3 SRGAP3 SYTL2 TGFB1 THEMIS TMX4 TNFAIP3 TSC22D3 TSPAN32 TTN TXNIP
XAF1 XCL1 YPEL3 ZNF217

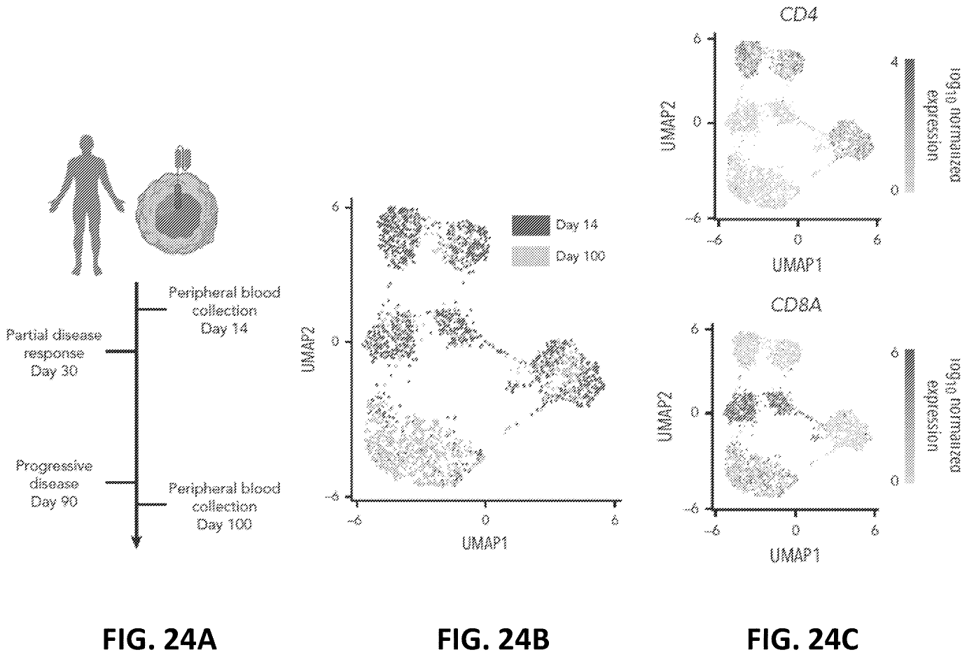

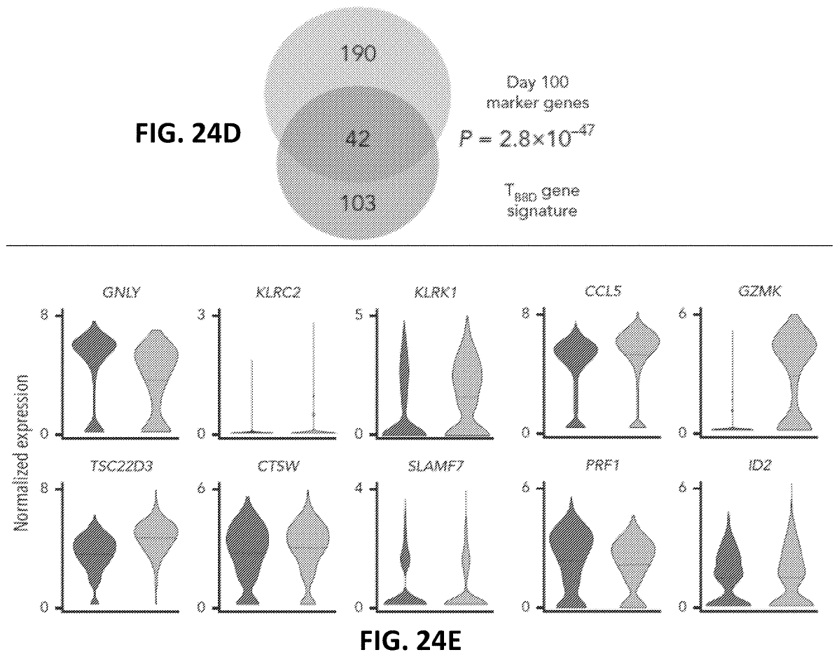

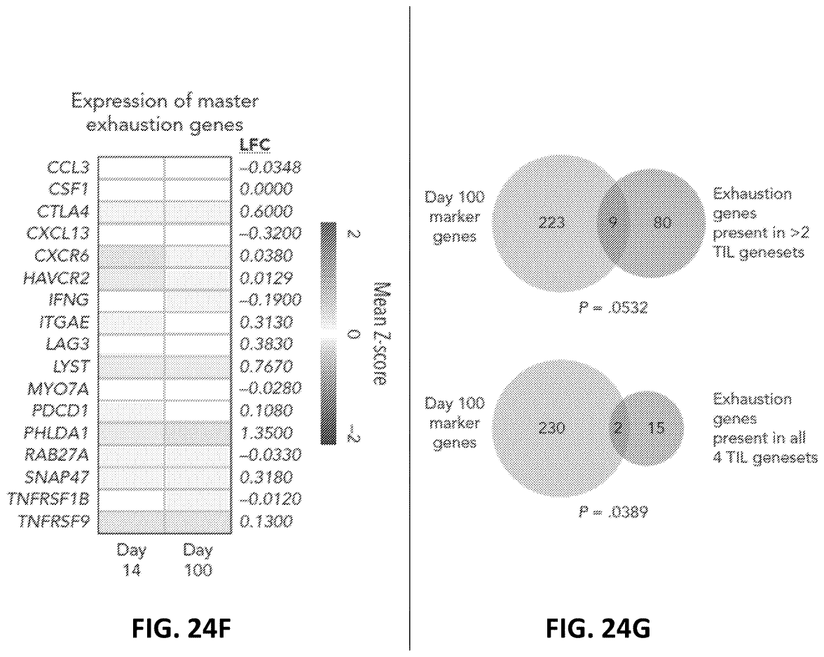

To determine whether this transcriptional state developed in CAR T-cell products that failed to induce remission in patients, circulating CAR T cells collected from a patient with diffuse large B-cell lymphoma who received tisagenlecleucel, a 41BB-based CD19 CAR T-cell product, were analyzed. This patient had a partial response 1 month after treatment but progressive disease at 3-month evaluation, a common clinical scenario that leads to persistent CAR stimulation. Circulating T cells at the time of partial antitumor activity (day 14) and after therapeutic failure (day 100; FIG. 24A) were evaluated. Day-14 CAR+ T cells clustered separately from day-100 cells, reflecting a transition in transcriptional identity (see e.g., FIG. 24B). Although the day-14 sample contained both CD4 and CD8 cells, the day-100 sample was composed almost entirely of CD8 cells (see e.g., FIG. 24C). The overlap between the T_{BBD} signature and the genes that defined the identity of day-100 CAR T cells (upregulated compared with day 14; n=232) was assessed and significant overlap between these 2 gene sets (P=2.8×10-47; FIG. 24D) was observed. Furthermore, 8 of the top 10 drivers of the T_{BBD} signature were expressed at higher levels in day-100 cells (see e.g., FIG. 24E). To assess whether day-100 cells also acquired features of exhaustion, expression of the exhaustion genes defined from TIL data sets was profiled. Although expression of 12 of these 17 genes were higher in day-100 cells, these differences were very modest (see e.g., FIG. 24F) and mirror the changes in exhaustion-associated gene expression observed in vitro (see e.g., FIG. 13C). Minimal overlap was observed between day-100 marker genes and TIL exhaustion signatures (see e.g., FIG. 24G), suggesting that failing 41BB-based CAR T cells evolve a transcriptional profile that more closely resembles the T_{BBD} gene profile than an exhausted gene profile.

### Dysfunctional 19/BB Cells Reactivate FOXO3

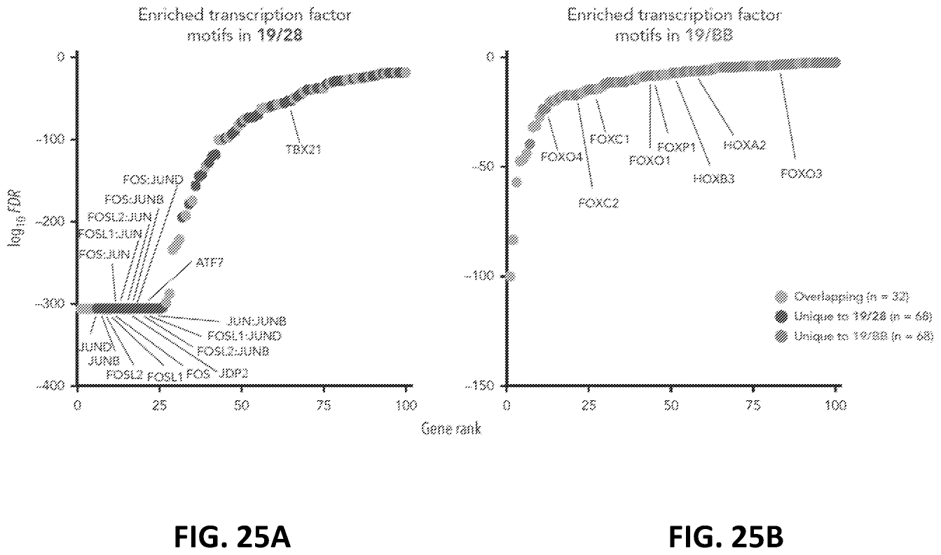

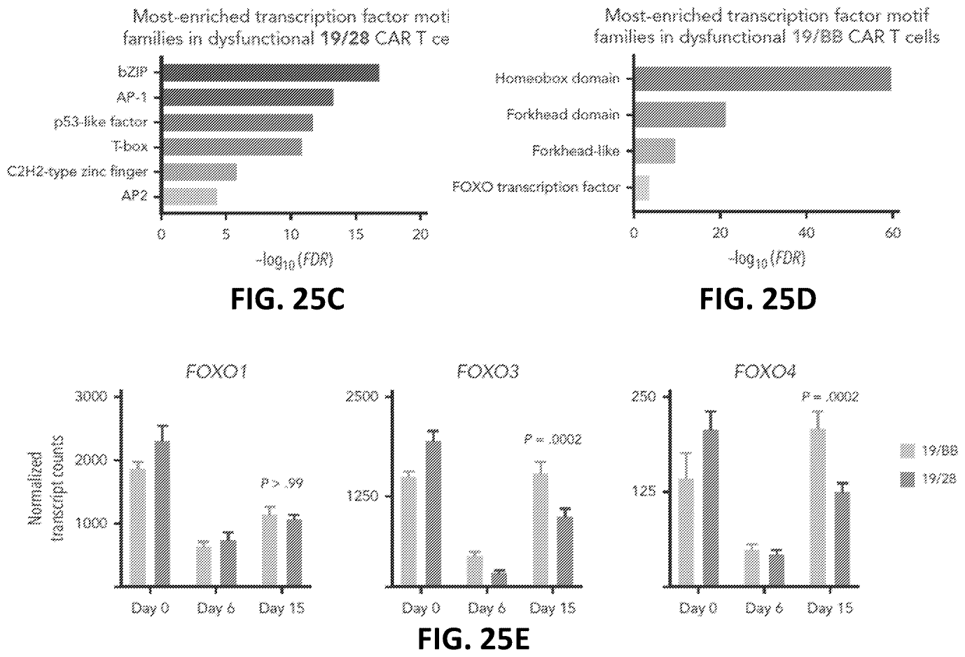

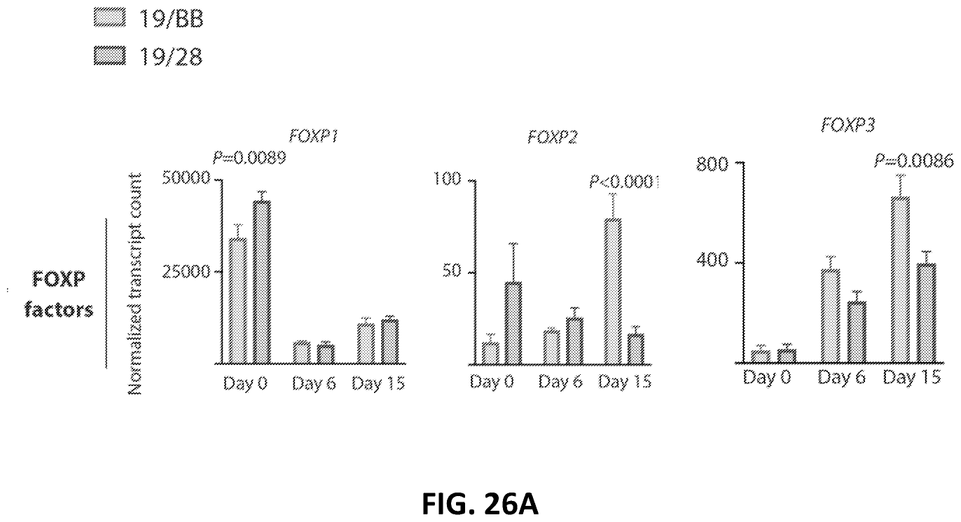

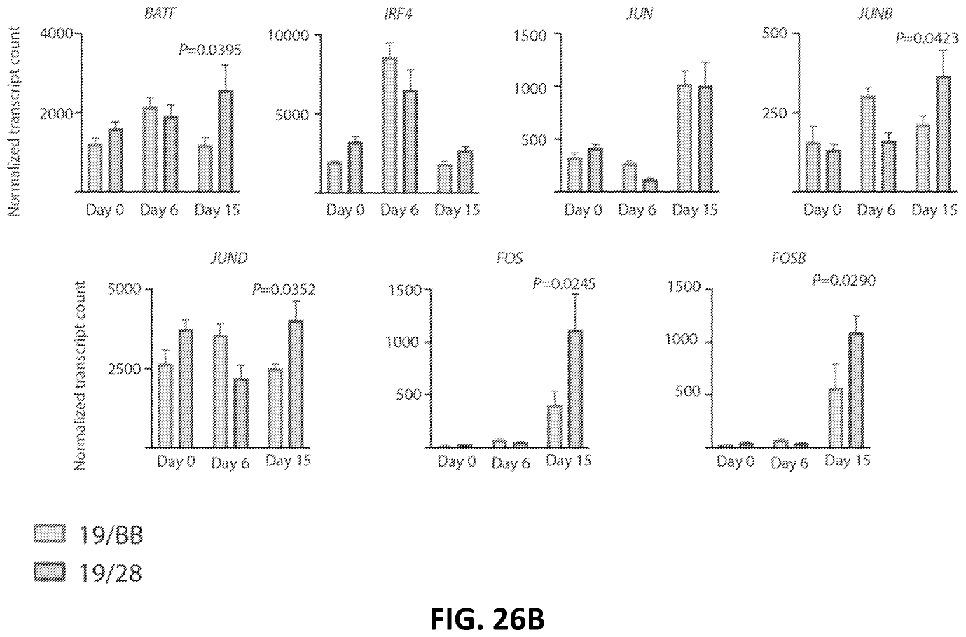

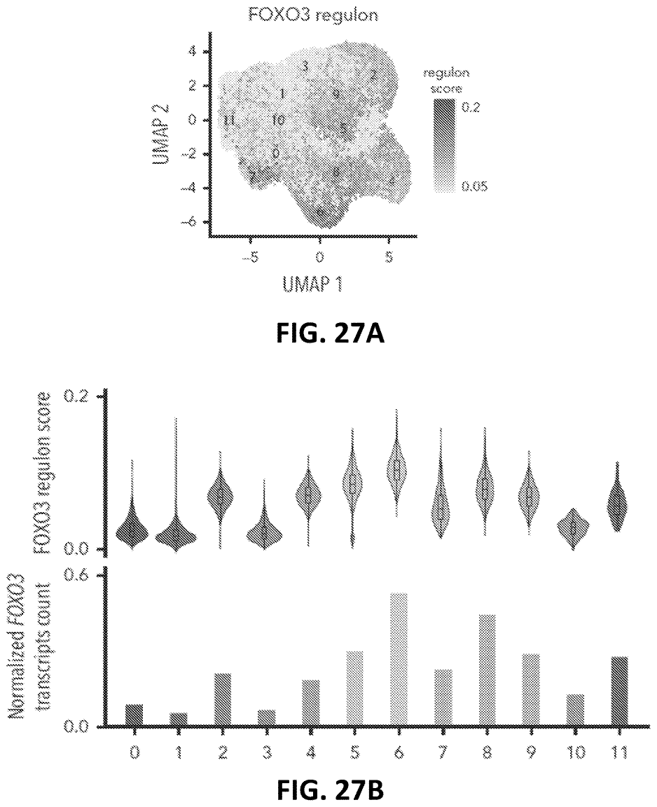

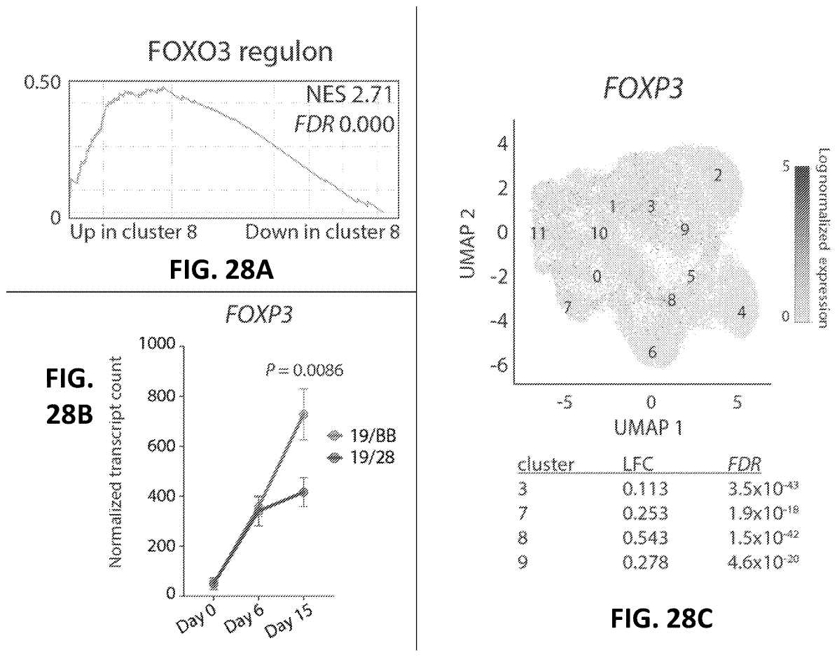

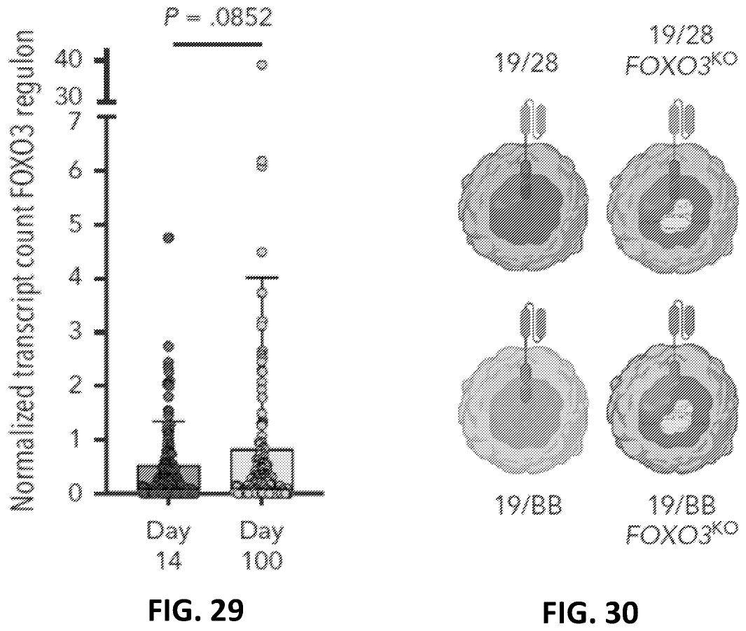

To identify the pathways responsible for promoting the development of this transcriptionally unique state, how transcription factor binding site accessibility evolved as 19/28 and 19/BB cells progressed from resting to dysfunctional was investigated. Although many of the sites (32/100) with increased accessibility were shared between 19/28 and 19/BB cells, more (68/100) were specific to each. As has been demonstrated previously, dysfunctional 19/28 cells had increased accessibility for Jun:Fos (AP1) binding (see e.g., FIG. 25A). In contrast, 19/BB cells opened homeobox (HOX) and forkhead box (FOX) sites (see e.g., FIG. 25B). Pathway analysis of the unique binding sites observed for each group confirmed that dysfunctional 19/28 cells were enriched for binding of basic leucine zipper domain-containing factors (which includes many proteins including AP1, see e.g., FIG. 25C). Dysfunctional 19/BB cells were enriched for HOX and FOX sites, with some specificity for FOXO sites (see e.g., FIG. 25D). Interrogation of the RNAseq data revealed nearly no expression of any HOX genes in day-15 cells but a resurgence in FOXO (see e.g., FIG. 25E) and some FOXP (see e.g., FIG. 26A) factors at day 15, which was more significant for 19/BB cells. The data also confirmed higher expression of basic leucine zipper domain factors in dysfunctional 19/28 cells (see e.g., FIG. 26B). Whether the increased binding site accessibility and transcript quantity was accompanied by increased FOXO activity was investigated. To do this, SCENIC, a tool designed to interrogate activity of transcription factor gene regulatory networks using scRNAseq data, was used. High activity of the FOXO3 regulon (target network) was observed in several clusters (2, 4, and 5-8; FIG. 27A). Quantification of regulon scores, reflecting quantity of FOXO3 target gene expression, and average FOXO3 transcript counts demonstrated that both were highest in cluster 6, composed entirely of day-0 cells and consistent with FOXO3's known role in maintaining T-cell homeostasis, and cluster 8, indicating high FOXO3 activity in dysfunctional 19/BB cells (see e.g., FIG. 27B). Gene set enrichment analysis of cluster-8 marker genes further confirmed enrichment of the FOXO3 regulon (normalized enrichment score, 2.71; false discovery rate, 0; see e.g., FIG. 28A). A key target of FOXO3 is FOXP3, responsible for directing development of regulatory T cells (T_{reg}). In addition to higher expression of CD25 (a marker of T_{regs}) in dysfunctional 19/BB cells (see e.g., FIG. 7), higher expression of FOXP3 transcripts in these cells was observed (see e.g., FIG. 28B). Evaluation of the single-cell data revealed that although FOXP3 transcripts were detectable in several clusters, they were highest in cluster 8 (see e.g., FIG. 28C). These data validate increased FOXO3 activity using both transcript and protein expression of a known FOXO3 target in dysfunctional 19/BB cells. Next, FOXO3 target gene expression was evaluated in peripheral blood CAR+ T cells and a trend toward increased activity was observed in day-100 cells (P=0.0852; FIG. 29). These data demonstrate that as 19/1B cells become dysfunctional they reactivate the transcription factor FOXO3.

### Disruption of FOXO3 Improves Function of 19/BB Cells

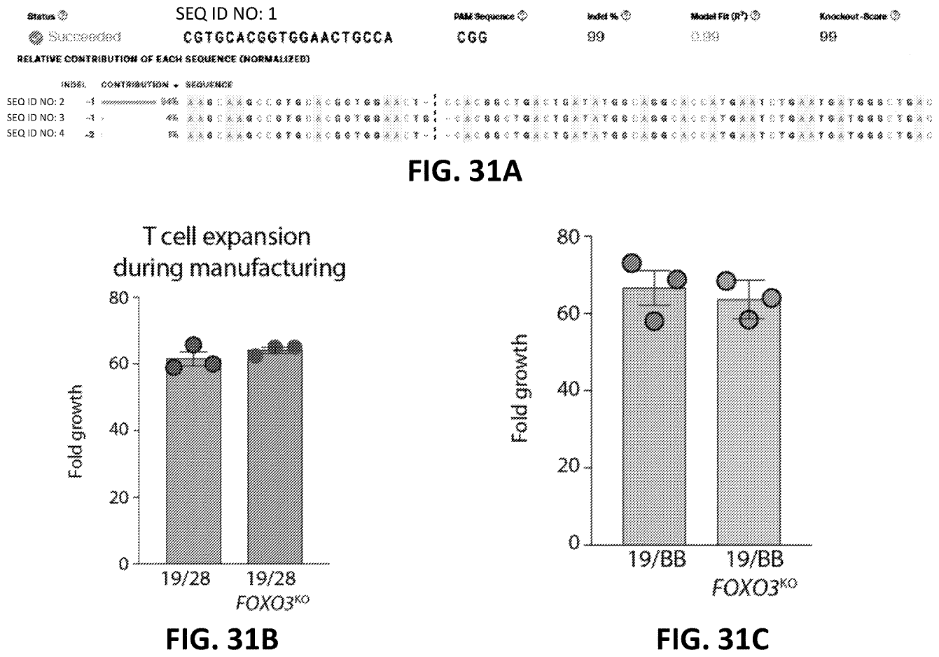

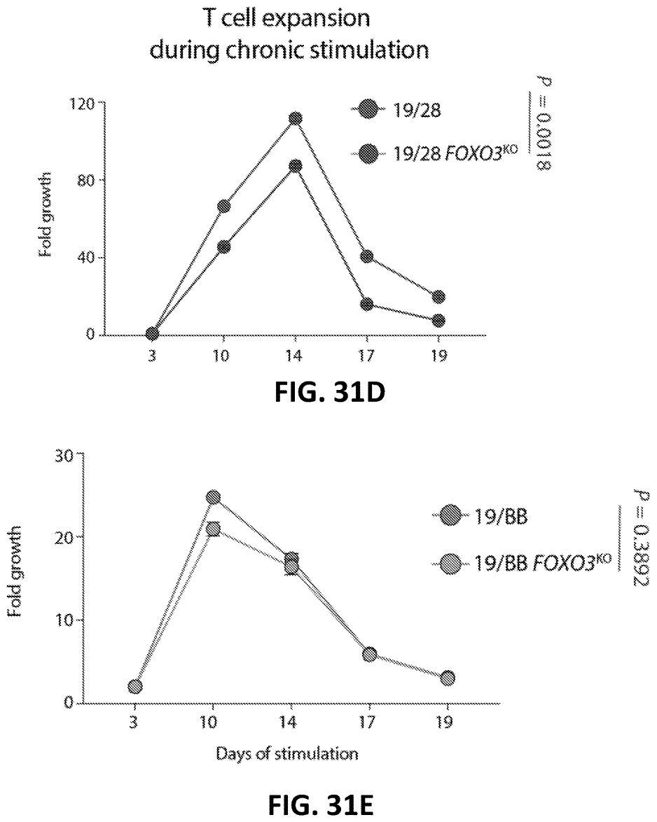

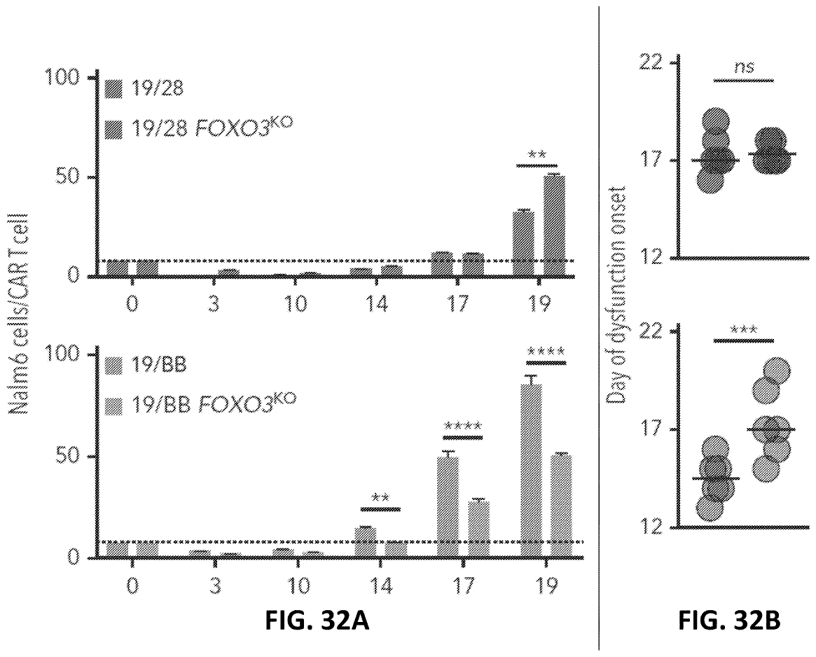

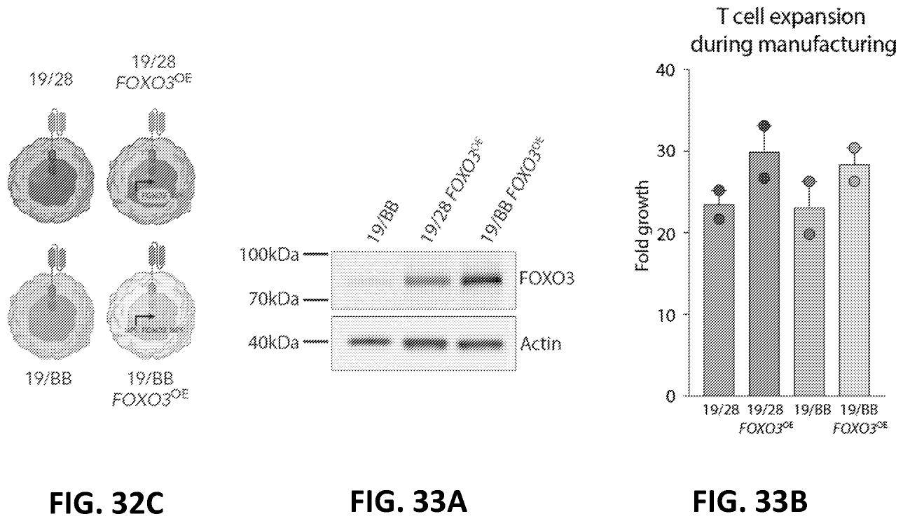

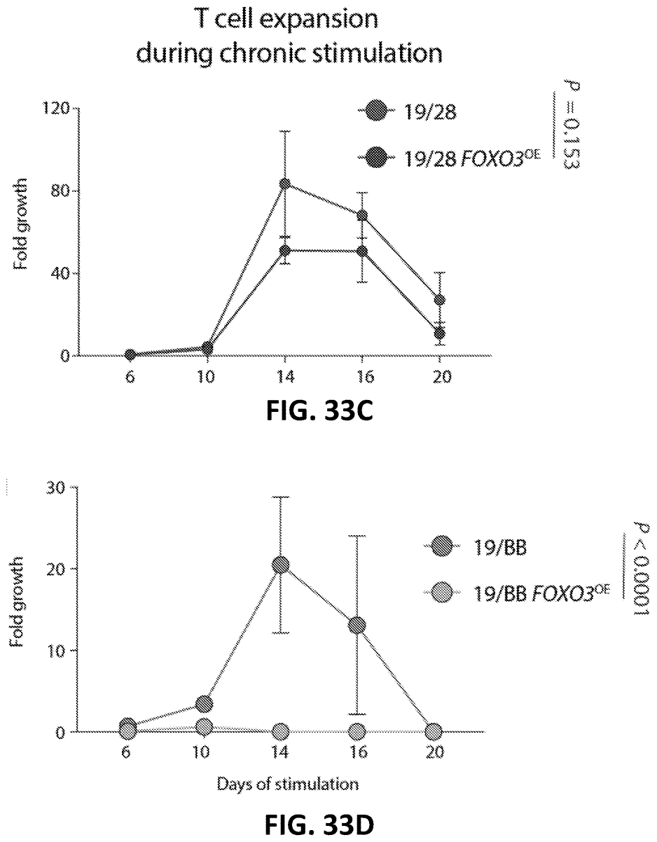

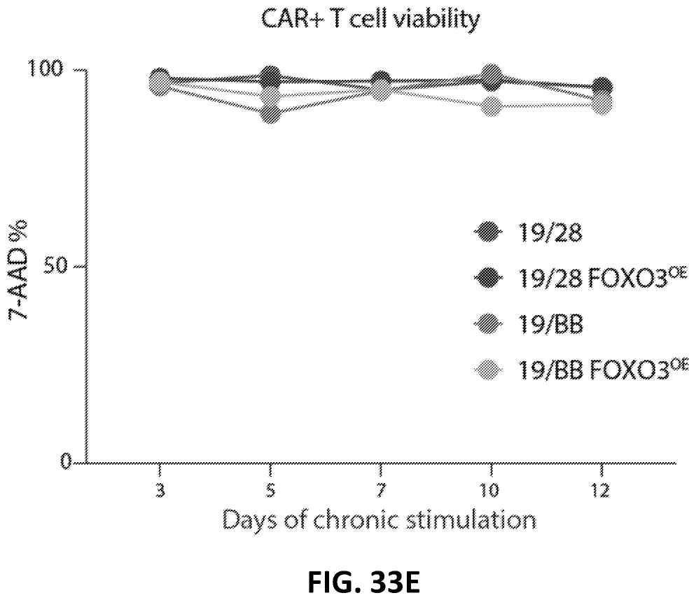

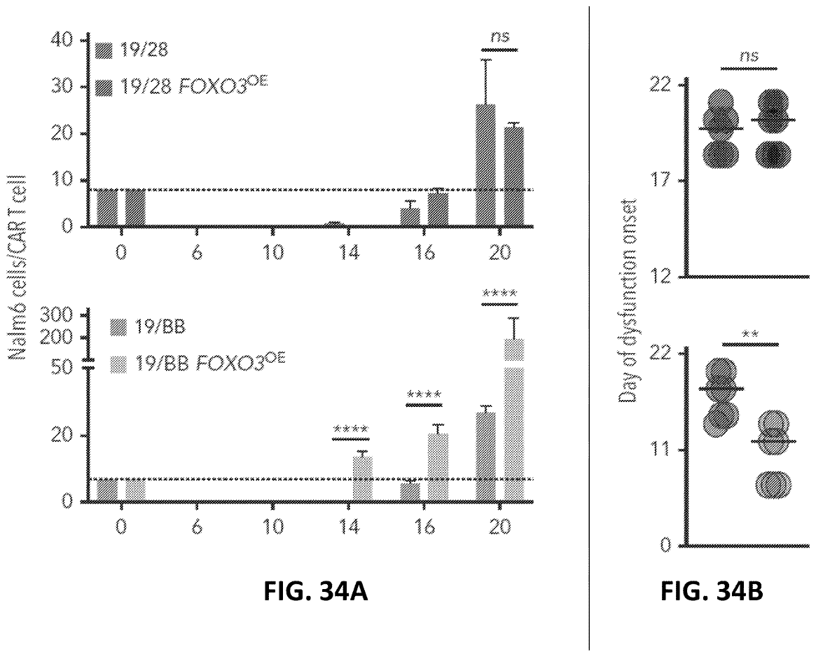

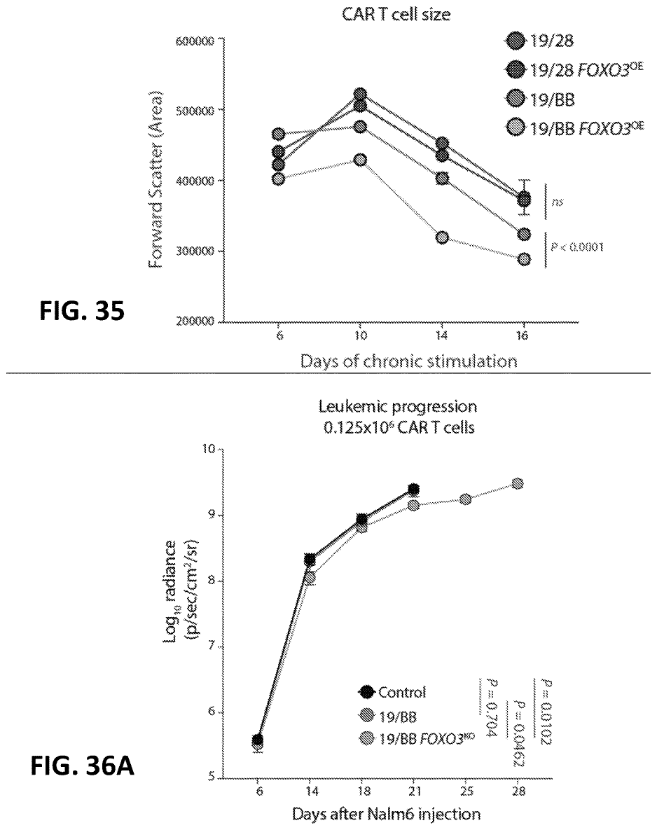

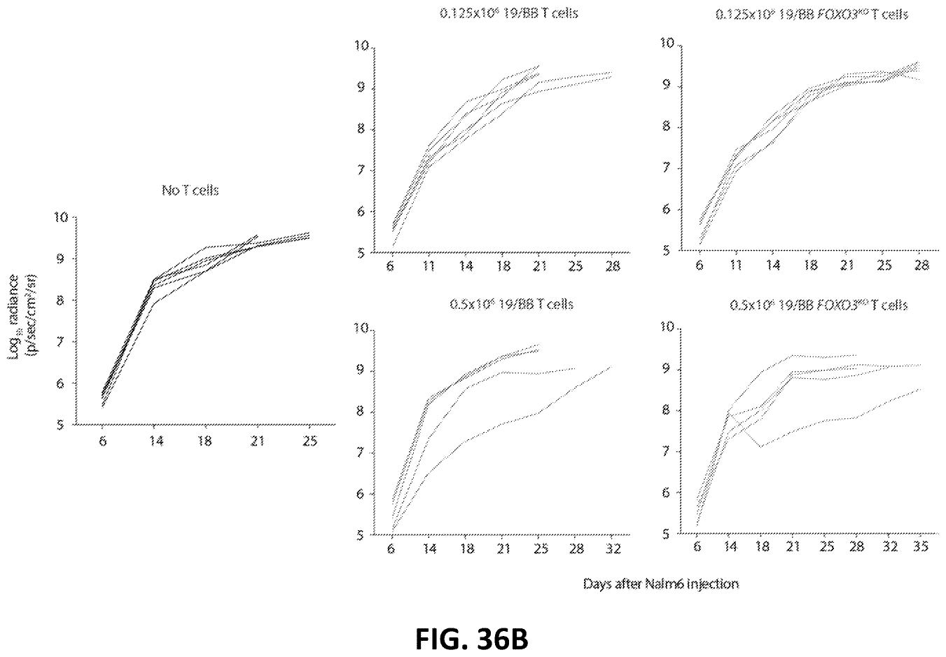

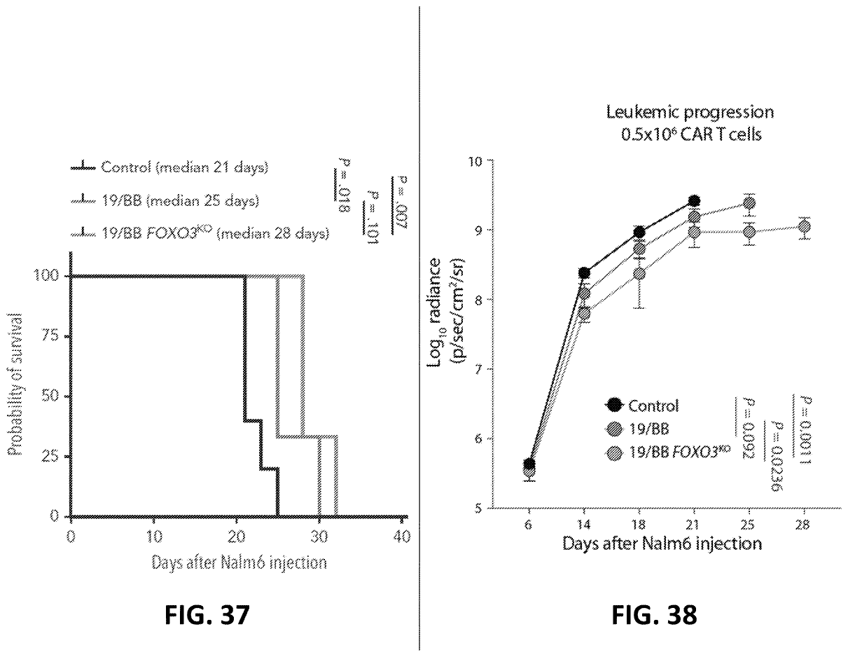

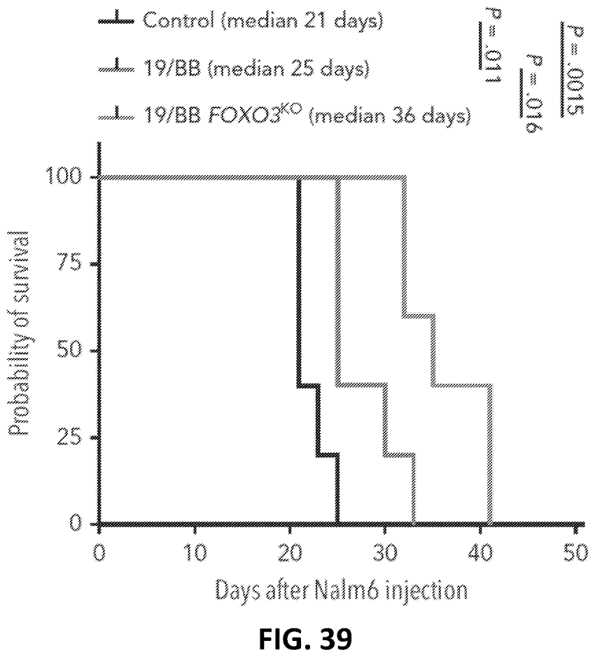

To determine whether FOXO3 inhibited 19/1B cell function, genomic FOXO3 was disrupted in 19/28 or 19/BB cells (see e.g., FIG. 30). This resulted in >90% gene disruption and had no impact on T-cell manufacturing (see e.g., FIG. 31A-FIG. 31C). These cells were subjected to the chronic in vitro stimulation assays and FOXO3 knockout (FOXO3^{KO}) was found to have minimal impact on CAR T-cell expansion (see e.g., FIG. 31D-FIG. 31E). In contrast, FOXO3^{KO} improved antitumor function of 19/BB cells but had no change in function of 19/28 cells, with a resultant delay in onset of dysfunction (see e.g., FIG. 32A-FIG. 32B). To substantiate the role of FOXO3 in dysfunction, the CAR constructs were engineered to include a transgenic FOXO3 (see e.g., FIG. 32C). This led to overexpression (OE) of FOXO3, which resulted in a minor improvement in cell expansion during manufacturing (see e.g., FIG. 33A-FIG. 33B). In chronic stimulation cultures, FOXO3^{OE} did not affect 19/28 cell expansion but dramatically inhibited 19/BB cell expansion (peak expansion from baseline of 22.8-fold compared with 3.2-fold; P<0.0001; FIG. 33C-FIG. 33D). 19/BB FOXO3^{OE} did not undergo more cell death (see e.g., FIG. 33E), indicating that this lower cell count was because of suppressed expansion. This was accompanied by a rapid loss of tumor control for 19/1B cells (see e.g., FIG. 34A) and much earlier onset of dysfunction (see e.g., FIG. 34B). 19/BB FOXO3^{OE} cells were significantly smaller in size throughout chronic stimulation (see e.g., FIG. 35), suggestive of a less-activated cell state. The impact of FOXO3^{KO} on 19/BB cells in vivo was then evaluated. To mimic conditions of chronic stimulation, a validated stress model was used in which NOD-SCID/γ^{-/-} mice were engrafted with 10⁶ Nalm6 cells and then given subtherapeutic doses of CAR T cells 7 days later. In the first model 1.25×10⁵ CAR T cells were delivered and FOXO3^{KO} had a modest impact in improving antileukemic activity (see e.g., FIG. 36A-FIG. 36B) and animal survival (see e.g., FIG. 37). In a second model 5×10⁵ CAR T cells were delivered and significantly enhanced antitumor function of FOXO3^{KO} cells was observed (see e.g., FIG. 38 and FIG. 36B). At this higher dose, FOXO3^{KO} 19/BB cells enabled a more impactful prolongation of animal survival (25 days compared with 36 days; P=0.0162; see e.g., FIG. 39). Collectively, these data indicate that FOXO3 specifically suppresses function of 41BB-based CAR T cells.

### DISCUSSION

Demonstrated herein is that 41BB-based CARs direct a unique state of T-cell dysfunction that is, in part, reliant on FOXO3. A notable attribute of dysfunctional 19/BB cells is expression of genes encoding NK receptors, a finding that has been observed in various models of T-cell exhaustion. Interestingly, 2 recent reports identified a novel preterminal exhausted state (termed TKLR) (see e.g., Daniel et al. (2022) Nat Immunol. 23(11):1614-1627 and Giles et al. (2022) Nat Immunol. 23(11):1600-1613). These cells also expressed Gzma Gzmk, Cc15, and Id2 and lacked expression of terminal exhaustion markers Pdcd1, Lag3, and Tox, reflecting high similarity to the T_{BBD} transcriptional signature defined here. Whether TKLR and dysfunctional 19/BB cells are a similar intermediate-exhausted lineage is not clear; dysfunctional 19/BB cells lack expression of CX3CR1, a defining marker of TKLR cells. Whether TKLR development is dependent on 41BB or instead relies on a shared independent pathway is the focus of ongoing studies. Intriguingly, both studies found that TKLR cells were a fraction of the bulk exhausted T-cell population, whereas herein the majority of cells present at day 15 of chronic 19/BB cell stimulation bear the T_{BBD} gene signature. If these cell states are indeed dependent on 41BB, this higher frequency may reflect the increased strength and duration of 41BB activation from CARs as opposed to natural costimulatory signals resulting from chronic infection.

A recent study similarly interrogated the trajectory of chronically stimulated 41BB-based CAR T cells (see e.g., Good et al. (2021) Cell. 184(25):6081-6100.e26). Although the authors also observed expression of NK receptors, they did not identify the same transcriptional features or regulatory pathways as described herein. Whether this is a result of a context dependence to this dysfunctional circuitry (their CARs targeted low-abundance mesothelin on pancreatic cancer, whereas the model herein targets high-abundance CD19 on leukemia) remains unknown. The authors also observed increased expression of classical exhaustion markers in dysfunctional as compared with resting CAR T cells, as have others. Herein, an increase in exhaustion marker expression was observed as these cells progress from resting to dysfunctional, but this expression is modest compared with CD28-based CAR T cells and is restricted to a distinct, smaller subset of dysfunctional cells. These data highlight that low-level expression of exhaustion markers may not be the same as commitment to the classical exhaustion program; indeed, dysfunctional 19/BB cells were found to have closed chromatin at the PDCD1 locus, which would preclude defining these cells as classically exhausted. Notably, expression of NK receptor genes was not observed in CD28-based CAR T cells, suggesting that this is not a broad feature of CAR-driven dysfunction but is dependent on costimulatory structure. FOX factors have broad roles in an array of cellular functions across tissue types. By inducing FOXP3 expression, FOXO3 promotes differentiation of induced T_{regs}. The data herein demonstrate an enrichment of CD25+ and FOXP3+ cells in dysfunctional 19/BB cells. Two recent reports identified that enrichment of T_{regs} is associated with clinical failure of CAR T cells, directly implicating a role for this pathway in limiting antitumor efficacy. These studies evaluated preinfusion and early postinfusion CD28-based CAR T cells; thus, the contribution of induced T_{regs} to the impaired functionality of 19/BB cells remains to be determined. FOXO3 also reportedly suppresses antiviral T-cell function by inhibiting the formation of durable memory in patients with HIV and in murine models of lymphocytic choriomeningitis. Of particular relevance to the findings herein, a distinct study found that Foxo3 deletion significantly improved T-cell control of chronic lymphocytic choriomeningitis virus infection, bolstering the findings herein by corroborating a role for FOXO3 in a distinct model of chronic antigen stimulation (see e.g., Sullivan et al. (2012) J Virol. 86(17):9025-9034). How FOXO3 exerts this suppressive function remains unclear, and of specific interest to the development of next-generation CAR therapies is the link between 41BB and FOXO3. Surprisingly, as observed herein, manipulation of FOXO3 did not impact T-cell manufacturing or the function of CD28-based CARs, both of which are driven by CD3/CD28 signaling.

These findings support the present disclosure that FOXO3's suppressive effect is dependent on 41BB and underscore that elucidating the relationship between 41BB, FOXO3, and activation of the TBBD dysfunction program is of importance. Although beneficial, disruption of FOXO3 resulted in modest delay in the onset of 41BB-based CAR T-cell failure. This is consistent with the improvements noted in other studies that disrupt T-cell transcription factors in an effort to improve CAR efficacy and suggests that an individual factor is unlikely to completely rescue CAR-driven T-cell dysfunction. Multiplexed manipulations that exploit vulnerabilities in distinct pathways of CAR-driven circuitry are likely to propel improvements in function. The study herein included clinical samples from 1 patient. Given the nature of the question, sequencing of cells undergoing chronic stimulation for a prolonged period was prioritized. Most studies evaluate CAR T cells collected between days 7 and 30 after infusion, or cells collected at later time points from patients with durable responses, a clinical setting in which CAR T cells are not chronically stimulated and do not persist longer. Described herein is the first study to report data on CAR T cells isolated months after infusion from a patient with progressive lymphoma, a scenario in which CAR T-cell persistence is highly limited beyond 30 days. Although this study demonstrated feasibility of evaluating these late-time point cells, this analysis required a significant volume of peripheral blood, which is not available for many patients enrolled on current banking protocols. Future studies will improve collection procedures to enhance the ability to evaluate these valuable tissues.

### Methods

### CAR T Cell Manufacturing and Chronic Stimulation Cultures

Lentiviral vectors were manufactured as previously described (see e.g., Singh et al. (2020) Cancer Discov. 10(4):552-567). PBMCs were procured from Miltenyi Biotec and CD4 and CD8 cells were purified using magnetic beads (Miltenyi Biotec) and combined at a 1:1 ratio. T cells were activated using CD3/CD28 stimulatory beads (DynaBeads; Thermo-Fisher) at a ratio of 3 beads/cell and incubated at 37° C. overnight. The following day, CAR lentiviral vectors were added to stimulatory cultures at a MOI of 2-4. Beads were removed after 6 days of stimulation, and cells were counted daily until growth kinetics and cell size demonstrated they had rested from stimulation. For all studies described, CAR constructs also encoded a truncated CD34 (tCD34) surface marker of transduction, separated from the CAR transgene by a P2A sequence. Both CD28 and 41BB-based CARs were composed of the FMC63 single chain variable fragment targeting CD19, CD8a hinge and transmembrane regions, followed by a costimulatory domain and a terminal CD3ζ signaling domain.

### General Cell Culture and Flow Cytometry

Unless otherwise specified, cells were grown and cultured at a concentration of 1×10⁶ cells/mL of standard culture media (RPMI 1640+10% FCS, 1% penicillin/streptomycin, 1% HEPES, 1% nonessential amino acids) at 37° C. in 5% ambient C02. All co-culture studies were performed at an effector cell to target cell ratio of 1:8, unless otherwise stated. Samples were stained with CD34 (BD, clone 581, #555824), CD4 (Biolegend, clone OKT4, #317444), CD8 (Invitrogen, clone SK1, #17-0087-42), PD1 (Biolegend, clone eh12.2h7, #329928), TIM3 (Invitrogen, clone F38-2E2, #17-3109-42), LAG3 (Biolegend, clone 11c3c65, #369314), CD62L (Biolegend, clone dreg-56, #304822), CD45RO (Biolegend, clone UCHL1, #204236) and 7-AAD (BD, #559925) in 100 ul FACS buffer (2% FBS in PBS), washed once with the same buffer and analyzed on the Attune NxT Flow Cytometer (ThermoFisher). GFP+ Nalm6 and GFP-CD34+ CAR T cells were gated and analyzed using FlowJo v9 or 10 (BD Biosciences).

### CyTOF

Mass cytometry was performed as previously described (see e.g., Berrien-Elliott et al. (2022) Sci Transl Med. 14(633):eabm1375). Briefly, isolated CAR+ T cells were live/dead stained with a short pulse of cisplatin and surface stained for 30 minutes at room temperature. Cells were then washed and fixed overnight at 4° C. with fix/perm buffer (eBiosciences). Intracellular staining was performed the following day at 4° C. for 1 hour. Cells were barcoded according to manufacturer's instructions (Fluidigm). Cells were washed and suspended in PBS containing 2% paraformaldehyde with Cell-ID Intercalator-IR. Mass cytometry data was collected on a Helios mass cytometer and analyzed using Cytobank (Beckman Coulter).

### Bulk RNA and ATAC Sequencing and Data Analysis

RNA sequencing was performed on samples derived from 1 donor for day 0 samples and 4 donors for day 6 and 15 samples. Each assay was performed in technical triplicate and one sample from both day 15 samples was excluded due to poor RNA quality, resulting in n=3 samples at day 0, n=12 samples at day 6 and n=11 samples at day 15. Total RNA was extracted using Qiazol (Qiagen) and recovered by RNA Clean and Concentrator spin columns (Zymo). Samples were prepared according to library kit manufacturer's protocol, indexed, pooled, and sequenced on an Illumina NovaSeq 6000. Basecalls and demultiplexing were performed with Illumina's bcl2fastq2 software. RNA-seq reads were then aligned and quantitated to the Ensembl release 101 primary assembly with an Illumina DRAGEN Bio-IT onpremise server running version 3.9.3-8 software. All gene counts were then imported into the R/Bioconductor package EdgeR13 and TMM normalization size factors were calculated to adjust for samples for differences in library size. The TMM size factors and the matrix of counts were then imported into the R/Bioconductor package Limma. Weighted likelihoods based on the observed mean-variance relationship of every gene and sample were then calculated for all samples and the count matrix was transformed to moderated log 2 counts-per-million with Limma's voomWithQualityWeights. Differential expression analysis was then performed to analyze for differences between conditions and the results were filtered for only those genes with Benjamini-Hochberg false-discovery rate adjusted p-values less than or equal to 0.05. Further analysis was performed using Partek Flow (Partek Inc). Geneset Enrichment Analysis was done using GSEA v4.1.0.

ATAC sequencing was performed on samples derived from 2 independent donors, also performed in technical triplicate resulting in n=6 samples for each time point. Omni ATAC-seq libraries were made as previously described (see e.g., Corces et al. (2017) Nat Methods. 14(10):959-962). Briefly, nuclei were isolated from 50,000 sorted CART19 cells, followed by the transposition reaction using Tn5 transposase (Illumina) for 30 minutes at 37° C. with 1000 rp mixing. Purification of transposed DNA was completed with DNA Clean and Concentrator (Zymo) and fragments were barcoded with ATAC-seq indices. Final libraries were double size selected using AMPure beads prior to sequencing. Paired-end sequencing (2×75 bp reads) was carried out on an Illumina NextSeq 500 platform. Adapters were trimmed using attack (version 0.1.5), and raw reads were aligned to the GRCh37/hg19 genome using bowtie with the following flags: --chunkmbs 2000 --sam --best --strata-ml-X 2000.15 MACS2 was used for peak calling with an FDR cutoff of 0.05. Downstream analysis and visualization, including transcription factor motif analysis, was done using Partek Flow (Partek Inc).

### Single Cell RNA Sequencing

CAR T cells from chronic stimulation cultures were isolated using flow-based sorting as described. For clinical samples, frozen vials of peripheral blood from a patient who underwent CAR T cell therapy and experienced a transient partial response followed by disease progression were gently thawed, counted, and dead cells were removed (Dead Cells Removal kit, Miltenyi, #130-090-101). Resulting cell samples had viabilities of 92-98% and were stained using an anti-FMC63 antibody (Acro Biochemicals, clone Y45, #FM3-HPY53) and then enriched for CAR+ cells using flow-based sorting. These cells were then processed using the 10× Genomics Chromium Single Cell V(D)J Reagent Kits (10× Genomics, PN-1000006, PN-1000020, PN-120262) to generate single-cell emulsions for barcoding, reverse transcription, and cDNA amplification. Immediately following these steps, 10× barcoded fragments were pooled and attached to standard Illumina adaptors to generate a barcoded single-cell RNA library. Sequencing libraries were quantified by qPCR before sequencing on the Illumina platform using HiSeq 4000 instrument. Cell Ranger's count pipeline v6.1.1 was applied to align reads and quantify gene expression of individual samples. Downstream single-cell analysis was performed using Seurat package v4.0.5 within the R programming environment v4.1.2. Lower bound for the number of genes in individual cells was chosen based on binary logarithm distribution and was set to 9.8 for the dataset with chronic stimulation samples and 10.5 for the dataset with clinical samples. Additionally, cells with more than 7,500 genes for the dataset with chronic stimulation samples and 4,000 genes for the dataset with clinical samples were filtered out. The percentage of mitochondrial counts was calculated for every cell, and only cells with mitochondrial percentage less than 10% were used in further analysis. Filtered matrices were normalized using a scaling factor of 10,000 and centered. Two sources of unwanted variation, total number of counts and percentage of counts belonging to mitochondrial genes, were regressed using a linear model. Within the datasets samples were combined using the harmony batch correction function. UMAP dimensional reduction and shared nearest neighbor graph were calculated on harmony corrected PCA embeddings using 20 principal components. Number of principal components was selected based on the elbow plot. Graph-based clustering was performed on the reduced data. Differential expression analysis between clusters was performed using the MAST algorithm of Seurat R package, p value adjustment was done using Bonferroni correction. CD8+ T cells from the chronic stimulation dataset were processed with Monocle2 pseudotime analysis pipeline. Seurat object were converted into CellDataSet object and used as an input. Differentially expressed genes between the clusters were identified with generalized linear model MAST, filtered by a significance level of p adjusted<0.05 and used for cell ordering. Dimensionality reduction was performed with DDRTree method. The cells from the day 0 were set as the root of the trajectory. Single-cell regulatory network analysis was performed with pySCENIC. First, Seurat objects with raw filtered counts were converted into AnnData files via SaveH5Seurat and Convert functions from the SeuratDisk package. Next, the adjacency matrix for transcription factors (hg38) and its targets were created using the GRNBoost2 algorithm. Motif analysis was performed using the cisTarget database. Cellular enrichment for each regulon was calculated by the AUCell module with default thresholds. Visualization and downstream analysis of pySCENIC output were performed with the SCopeLoomR package.

### TBBD Gene Signature Development

Which genes were biomarkers, or specifically enriched, in day 15 19/BB bulk RNA data and cluster 8 was determined from scRNAseq data using Partek Flow. The overlap of each data set was used to generate the 145 gene signature. For these genes a “contribution score” was developed by multiplying the fold change of each gene relative to all other samples (5 samples from bulk RNAseq and 11 clusters for scRNAseq) from bulk and scRNAseq data. The 10 genes with the largest score were used for analysis in FIG. 24E.

### CRISPR/Cas9 Gene Editing

CRISPR sgRNAs were designed using the CRISPick tool from The Broad Institute and the sgRNA design tool from Integrated DNA Technologies (IDT). Cells were electroporated using the Lonza 4DNucleofector Core/X Unit. Triple Reporter Jurkat cells were electroporated using the SE Cell Line 4-D Nucleofector Kit, and primary T cells were electroporated using the P3 Primary Cell 4-D Kit (Lonza). For Cas9 and sgRNA delivery, a ribonucleoprotein (RNP) complex was first formed by incubating 5 μg of TrueCut Cas9 Protein V2 (Lonza) with 10 μg of sgRNA for 10 min at room temperature. Cells were washed twice with PBS (Gibco) and spun at 100×g for 10 min and resuspended at a concentration of 2-10×10⁶ cells/100 μL in the specified buffer. The RNP complex and 100 μL of resuspended cells were combined and electroporated. Pulse codes EO-115 and CV-104 were used for resting primary T cells and Jurkat cells, respectively. After electroporation, the cells were incubated in standard 10% RPMI for Jurkat cells and cytokine enriched 10% RPMI (5 ng/ml IL7 and 5 ng/ml IL15, both from Peprotech) for primary T cells for the duration of experiment. Primary T cells were stimulated after 18 hours using CD3/CD28 Dynabeads (Thermo-Fisher) at 1:3 cell to bead ratio and engineered with lentivirus 24 hours later. To determine efficiency of gene disruption, TIDE (Tracking of Indels by DEcomposition) analysis was used to detect knock out (KO) efficiency. Genomic DNA from electroporated cells was isolated (Qiagen DNeasy Blood & Tissue Kit) and 100-200 ng were PCR amplified using Q5 Hot Start High Fidelity 2× Master Mix (NEB) and 10 μM forward/reverse primers flanking the region of interest. Primers were designed such that the amplicon was at a target size ˜1 kb. PCR products were gel or column purified and sequenced, and trace files were analyzed using TIDE web tool to determine indel proportions. R2 values were calculated, reflecting goodness of fit after non-negative linear modeling by TIDE software.

### Xenograft Mouse Models

6-10 week old NOD-SCID-γc/^{-/-} (NSG) mice were obtained from the Jackson Laboratory and maintained in pathogen-free conditions. Animals were injected via tail vein with 1×10⁶ Nalm6 cells in 0.2 mL sterile PBS. On day 7 after tumor delivery, either 0.125×10⁶ or 0.5×10⁶ CAR+ T cells (WT or FOXO3^{KO}) were injected via tail vein in 0.2 mL sterile PBS. Animals were monitored for signs of disease progression and overt toxicity, such as xenogeneic graft-versus-host disease, as evidenced by >10% loss in body weight, loss of fur, diarrhea, conjunctivitis and disease-related hind limb paralysis. Disease burdens were monitored over time using the Xenogen IVIS bioluminescent imaging system, as previously described (see e.g., Singh et al. (2020) Cancer Discov. 10(4):552-567) and animals were sacrificed when radiance reached>3×10⁹ photos/sec/cm²/sr (5-log greater than background). To avoid skewing of radiance data, graphical representation for each group was stopped after death of the first animal in the group.

### Statistical Analysis

All comparisons between two groups were performed using either a two-tailed unpaired Student's t-test or Mann-Whitney test, depending on normality of distribution. Comparisons between more than two groups were performed by two-way analysis of variance (ANOVA) with Bonferroni correction for multiple comparisons. All results are represented as mean±standard error of the mean (s.e.m.). Survival data were analyzed using the Log-Rank (Mantel-Cox) test.

### Example 2: Regulation of the Transcriptional Repressor Bach2 Overcomes Tonic Signaling-Driven Car T Cell Dysfunction

This Example describes increasing BACH2 expression in CAR-T cells. BACH2 rescues phenotypes associated with T cell exhaustion and dysfunction in CARs with CD28 tonic signaling. Without added BACH2 expression, the CD28 tonic signaling cells (e.g., CD28^{hi}) quickly stop expanding and stop killing target ALL in co-culture experiments.

Unlike endogenous T cell receptors (TCRs), chimeric antigen receptors (CARs) have an intrinsic predisposition to initiate intracellular signaling in the absence of target antigen. This phenomenon, referred to as tonic signaling, has previously been shown to be a potent driver of T cell exhaustion when CARs contain the CD28 costimulatory domain. It was previously shown that, in contrast to CD28, CARs containing the 41BB costimulatory domain acquire enhanced functionality from tonic signaling (Singh et al. (2021) Nat Med. 27(5):842-850). These data indicate that the impact of tonic CAR signaling is directly dependent on the CAR costimulatory domain.

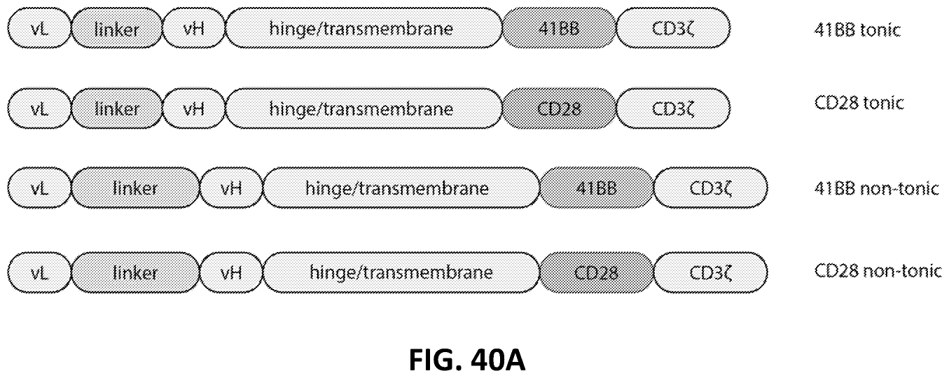

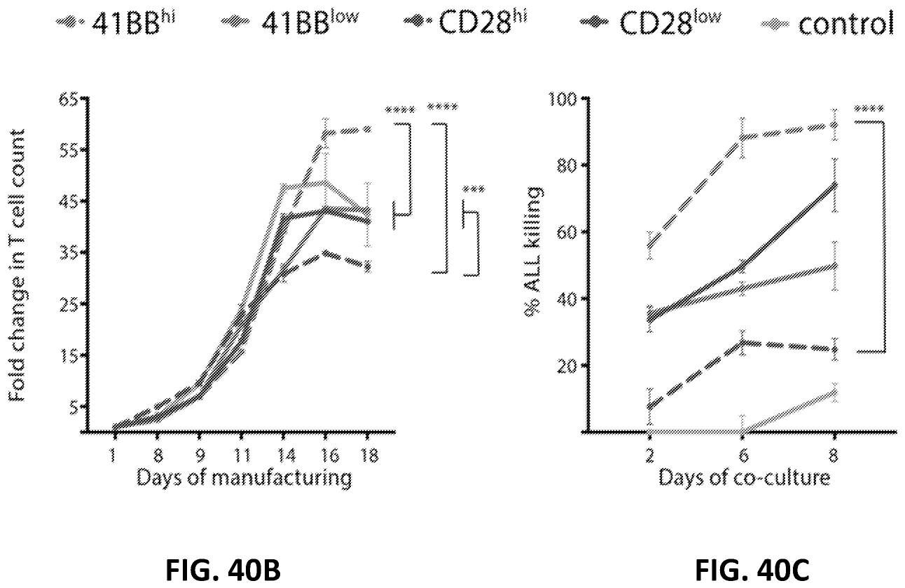

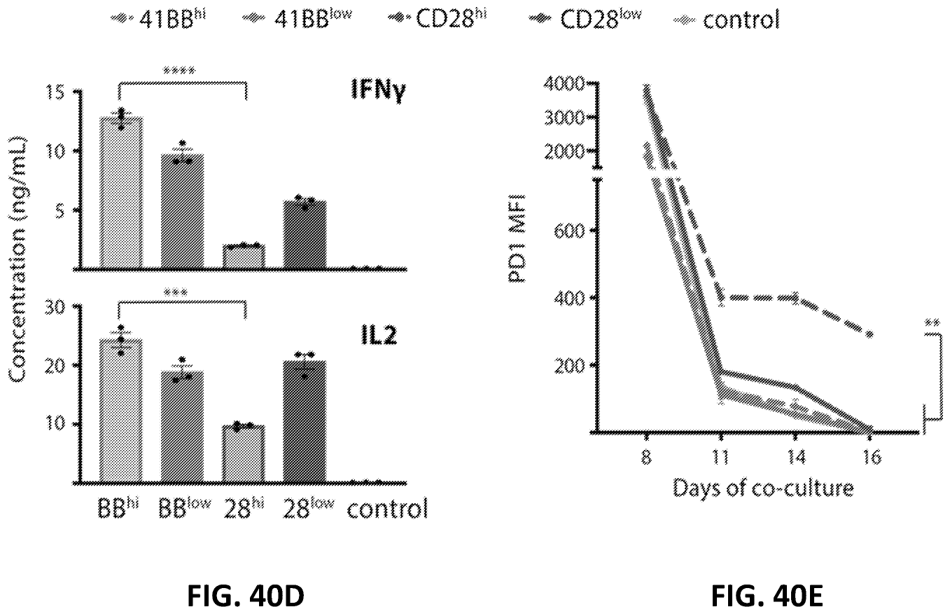

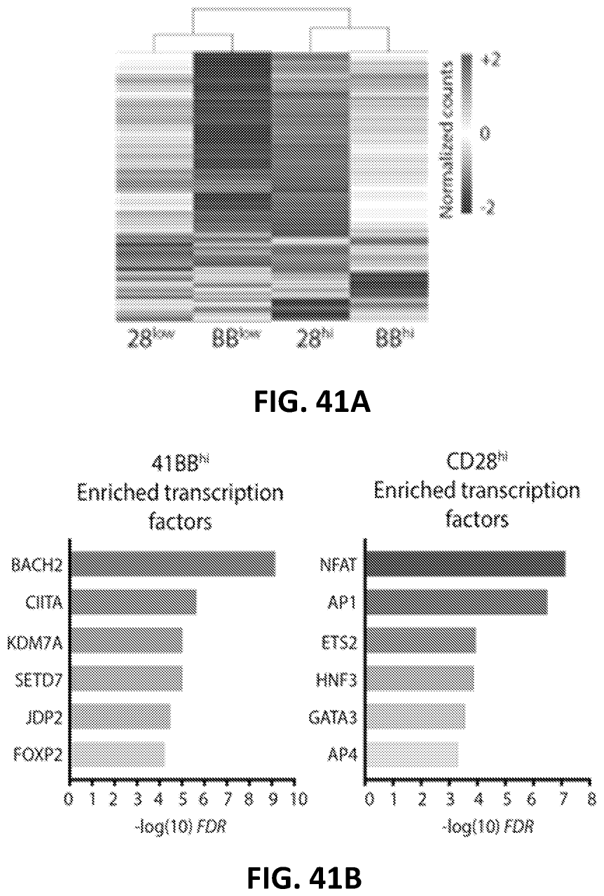

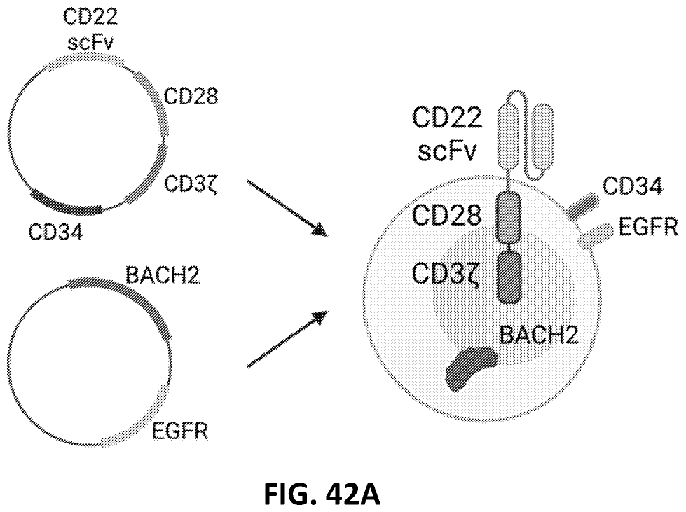

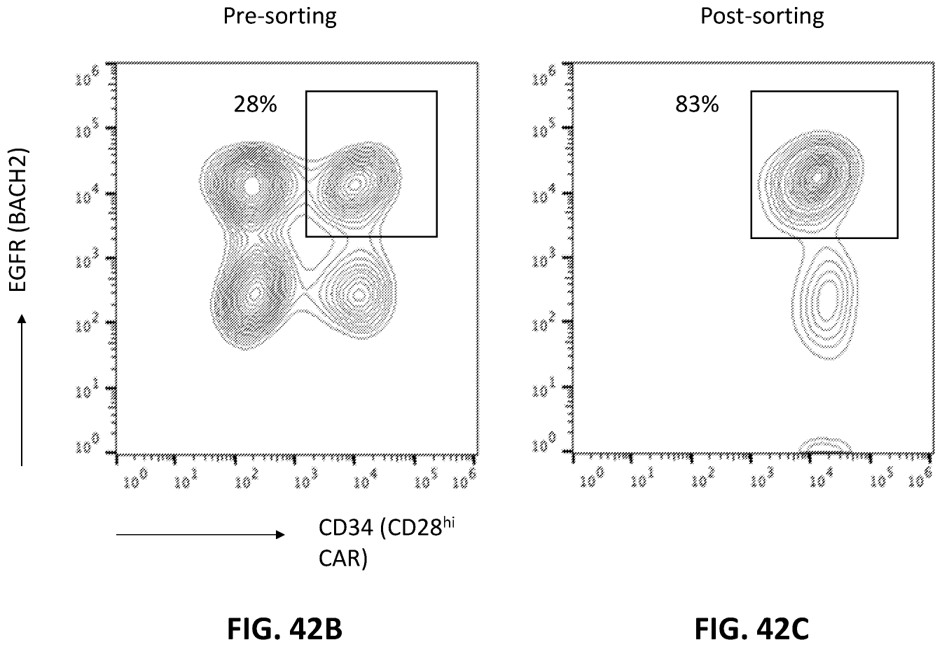

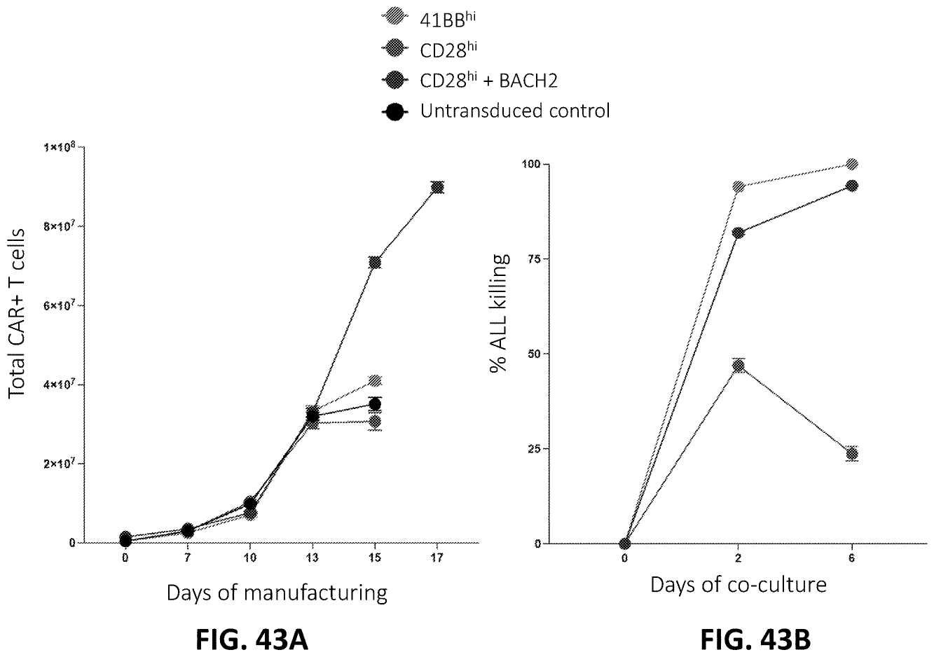

To understand the molecular regulators responsible for this divergent functionality, CARs were generated herein targeting CD22, an established immunotherapy target for B cell malignancies, that contained either CD28 or 41BB costimulatory domains and either did or did not signal tonically (22/28+, 22/BB+, 22/28−, 22/BB−, see e.g., FIG. 40A). Tonic signaling of CD28 was confirmed to drive impaired cytotoxic function, T cell expansion and cytokine secretion in response to antigen, while tonic 41BB improved all parameters of T cell function (see e.g., FIG. 40B-FIG. 40E). Bulk RNA sequencing of CAR T cell products at the conclusion of manufacturing, when the impact of tonic CAR signaling was predicted to be most impactful, revealed significantly more transcriptional activity in both tonic CARs (see e.g., FIG. 41A-FIG. 41B). Comparison of differentially expressed genes demonstrated that 22/28+ had high expression of targets of NFAT and AP1, transcription factors that promote T cell effector function but are also drivers of T cell exhaustion when persistently active. 22/BB+ instead demonstrated high activity of BACH2, a transcriptional repressor that has recently been shown to preserve naïve and memory cell states by antagonizing effector programs. 22/28+ cells that overexpressed BACH2 (22/28+_BACH2) were generated (see e.g., FIG. 42A-FIG. 42C) and this intervention completely rescued acute anti-tumor function, with cytotoxic activity of these cells equivalent to 22/BB+(see e.g., FIG. 43A-FIG. 43B). Mass cytometry revealed that 22/28+_BACH2 cells were phenotypically very similar to 22/BB+ cells at the conclusion of manufacturing and after stimulation with target cells, expressing high levels of granzyme B, perforin, IFNγ and markers of memory differentiation. Intriguingly, 22/28+_BACH2 cells expressed very high levels of the transcription factor TCF7, known to play a role in maintaining memory, suggesting that transgenic BACH2 expression play a broad role in lineage regulation.

To interrogate how BACH2 overexpression impacted long-term T cell function, these cells were chronically stimulated using an established in vitro protocol (Selli et al. (2023) STAR Protoc. 4(1):101954) in which co-cultures of CAR T cells and tumors are continually replenished with additional tumor to maintain a persistent and high antigen burden. Similar to previous studies, early function of 22/28+_BACH2 cells was significantly improved over 22/28+ but BACH2 overexpressing cells lost the ability to proliferate and kill tumor cells much earlier than either 22/28+ or 22/BB+. Phenotypic analysis revealed at time of cell failure revealed that almost all 22/28+_BACH2 cells expressed markers of a central memory phenotype (CD62L^{hi}CD45RO^{hi}).

Collectively, these data suggest that BACH2 overexpression can overcome tonic CAR signaling-induced dysfunction by antagonizing exhaustion programs but simultaneously “locks” these cells into a memory state with restrained effector function, thus limiting their long-term efficacy.

1. A composition for the treatment of cancer, the composition comprising a modified chimeric antigen receptor (CAR) T cell, wherein the modified CAR T cell expresses a reduced level of a FOXO transcription factor compared to an unmodified CAR T cell.

2. The composition of claim 1, wherein the FOXO transcription factor is FOXO1, FOXO3, or FOXO4.

3. The composition of claim 1, wherein the FOXO transcription factor is FOXO3.

4. The composition of claim 1, wherein the modified CAR T cell comprises a CD28 signaling domain.

5. The composition of claim 1, wherein the modified CAR T cell comprises a 41BB signaling domain.

6. The composition of claim 1, further comprising a FOXO inhibitor.

7. The composition of claim 1, wherein the modified CAR T cell is CRISPR edited to disrupt a FOXO transcription factor gene.

8. A method of treating cancer in a subject in need thereof, the method comprising:
administering to the subject an effective amount of a composition comprising a modified chimeric antigen receptor (CAR) T cell, wherein the modified CAR T cell expresses a reduced level of a FOXO transcription factor compared to an unmodified CAR T cell.

9. The method of claim 8, wherein the subject has leukemia or lymphoma.

10. A method for producing modified CAR T cells expressing a reduced level of a FOXO transcription factor compared to an unmodified CAR T cell, the method comprising:
culturing a population of CAR T cells;
introducing a composition to the population of CAR T cells that disrupts a FOXO transcription factor gene, yielding a population of modified CAR T cells; and
expanding the population of modified CAR T cells.

11. The method of claim 10, wherein the composition comprises a Cas protein and a single guide RNA (sgRNA) targeted to FOXO3.

12. The method of claim 10, wherein the population of CAR T cells is further modified to express a CD28 or 41BB signaling domain.

13. A composition for the treatment of cancer, the composition comprising a modified chimeric antigen receptor (CAR) T cell, wherein the modified CAR T cell expresses an increased level of BACH2 compared to an unmodified CAR T cell.

14. The composition of claim 13, wherein the modified CAR T cell comprises a CD28 signaling domain.

15. The composition of claim 13, wherein the modified CAR T cell is a CD22-targeting CAR T cell.

16. A method of treating cancer in a subject, the method comprising:
administering to the subject an effective amount of a composition comprising a modified chimeric antigen receptor (CAR) T cell, wherein the modified CAR T cell expresses an increased level of BACH2 compared to an unmodified CAR T cell.

17. A method for producing modified CAR T cells expressing an increased level of BACH2 compared to unmodified CAR T cells, the method comprising:
culturing a population of CAR T cells;
introducing a first vector comprising a nucleic acid encoding BACH2 to the population of CAR T cells; and
expanding the population of T cells.

18. The method of claim 17, further comprising introducing a second vector comprising a nucleic acid encoding CD28 to the population of CAR T cells.

19. The method of claim 18, wherein the first vector and the second vector each comprise a selection marker.

20. The method of claim 19, wherein the selection marker is a nucleic acid encoding EGFR or CD34.


SEQUENCE LISTING (4 sequences)

SEQ ID NO: 1 - DNA, 20 bp, Homo sapiens
  source 1..20: mol_type = genomic DNA
cgtgcacggtggaactgcca

SEQ ID NO: 2 - DNA, 74 bp, Homo sapiens
  source 1..74: mol_type = genomic DNA
aagcaagccgtgcacggtggaactccacggctgactgatatggcaggcaccatgaatctgaatgatgggctgac

SEQ ID NO: 3 - DNA, 74 bp, Homo sapiens
  source 1..74: mol_type = genomic DNA
aagcaagccgtgcacggtggaactgcacggctgactgatatggcaggcaccatgaatctgaatgatgggctgac

SEQ ID NO: 4 - DNA, 73 bp, Homo sapiens
  source 1..73: mol_type = genomic DNA
aagcaagccgtgcacggtggaactcacggctgactgatatggcaggcaccatgaatctgaatgatgggctgac